(19) United States
(12) Patent Application Publication
(10) Pub. No.: US 2024/0288424 A1
(43) Pub. Date: Aug. 29, 2024

(54) LABELING BIOLOGICAL PARTICLES USING ACOUSTOPHORESIS

(71) Applicant: THE UNITED STATES OF AMERICA, as Represented by the Secretary, Department of Health and Humag, Bethesda, MD (US)

(72) Inventors: Stephen S. Adler, Chevy Chase, MD (US); Noriko Sato, Potomac, MD (US); Peter Choyke, Rockville, MD (US)

(73) Assignee: THE UNITED STATES OF AMERICA, as Represented by the Secretary, Department of Health and Humag, Bethesda, MD (US)

(21) Appl. No.: 18/290,442

(22) PCT Filed: May 11, 2022
(86) PCT No.: PCT/US2022/028819

§ 371 Date: Nov. 13, 2023

Related U.S. Application Data
(60) Provisional application No. 63/191,103, filed on May 20, 2021.

(51) Int. Cl. G01N 33/569; B01L 3/00

(52) U.S. Cl. CPC G01N 33/56972; B01L 3/502761; B01L 2200/0652; B01L 2300/0663; B01L 2300/0864; B01L 2400/0436

(57) ABSTRACT

Methods and apparatus are described for labeling biological particles using acoustophoresis. In a washing stage, two adjacent substreams are passed through a region having a gradient in acoustic energy density, leading to particles being driven from one substream to the other. Washed bioparticles flowing in a medium are accumulated at a trapping site by an acoustic energy barrier while the medium passes by. Accumulated particles are discharged in batches and mixed with labeling particles. The mixture is incubated in a chamber, producing labeled biological particles, which are washed in another washing stage. Processing operations are automated e.g. on a microfluidics platform. Diagnostic and therapeutic applications are disclosed, covering a range of particle types and a range of labeling types. Exemplary biological particles include cells, antibodies, or fragments thereof. Exemplary labeling particles have detection functions (radioligand, magnetic particles, fluorophores), therapeutic functions (drugs), or binding functions (adhesion molecules).

### CROSS REFERENCE TO RELATED APPLICATIONS

This application claims benefit of U.S. Provisional Patent Application No. 63/191,103, filed May 20, 2021, which is incorporated in its entirety herein by reference.

### STATEMENT OF GOVERNMENT SUPPORT

This invention was made with Government support under contract number 75N91019D00024 by the National Institutes of Health, National Cancer Institute. The Government has certain rights in the invention.

### FIELD

This disclosure pertains to labeling biological particles using acoustophoresis.

### BACKGROUND

Cell labeling is an important technique in diagnostic and therapeutic applications. However, conventional procedures for labeling cells can be laborious, often requiring a sequence of multiple operations to be performed manually. For example, U.S. Pat. No. 10,556,916 to Sato et al. describes some conventional approaches for labeling bone marrow, EL4 lymphoblasts or other cells. These approaches generally involve manual handling of tubes for specimens and reagents, tabletop centrifuges for separation and, often, repetitive steps. Conventional approaches may require skilled operators and may be prone to human error.

For such reasons, conventional techniques have proven difficult to replicate in large numbers (difficult to “scale”) for trials or other large-scale applications. Accordingly, there is a need for improved technologies for cell labeling that can be integrated, automated, and scaled.

### SUMMARY

In brief, the disclosed technologies provide an integrated multi-stage technique for labeling bioparticles. Bioparticles can be washed, trapped, mixed, incubated, and washed again without manual intervention. Acoustophoresis can be used to implement one or more of the stages on a microfluidic platform or similar environment, e.g. driving bioparticles from one medium substream to another; or trapping bioparticles using an acoustic field. The disclosed technologies are suitable for integration and automation, leading to efficiency and high throughput as compared to conventional manually intensive techniques. The disclosed technologies are readily scalable, permitting many thousands of samples to be labeled conveniently for large scale studies or trials. The disclosed technologies can be configured for a wide range of applications, using a variety of bioparticle types and labels.

In certain examples, the disclosed technologies can be implemented as a method incorporating the following operations. (a) Bioparticles flowing in a first fluid pathway are washed from a first medium to a distinct buffer medium using acoustophoresis. (b) The washed bioparticles are trapped using acoustophoresis in a second fluid pathway. (c) A mixture of labeling particles and the trapped bioparticles (from the trapping) are incubated in a fluid chamber for a predetermined duration, to develop labeled bioparticles. (d) The mixture is washed using acoustophoresis in a third fluid pathway, and (e) the labeled bioparticles are extracted from the third fluid pathway.

In some examples, the bioparticles can include one or more of: antibodies, antibody fragments, cells, cell components, cell membrane components, extracellular vesicles, or proteins. The bioparticles can include T cells, regulatory T cells, or dendritic cells. The bioparticles can include mammalian cells or human cells. The bioparticles can include cellular proteins or cell membrane components. The bioparticles can include viable cells or dead cells. The labeling particles can include one or more of: an adhesion molecule, an adhesion molecule fragment, an antibody, an antibody fragment, a biotin ester, a drug, a dye, a fluorophore, a liposome, an inorganic nanoparticle, a nanotube, a magnetic label, a protein, a radioligand, or a small molecule drug. The labeling particles can include a carboxyfluorescein succinimidyl ester (CFSE), a fluorescent dye, a fluorophore conjugated antibody, or a fluorescent expression protein. The labeling particles can include an F-19 perfluorocarbon nanoemulsion or iron oxide nanoparticles. The labeling particles can include a DNA binding dye, a viability dye, or an ion indicator dye. The labeling particles can include Zr-89 oxine, In-111 oxine, or a Zr-89 conjugated antibody.

In additional examples, the method can include driving the bioparticles suspended in the first medium into the first fluid pathway as a first substream in contact with and in a same direction as a second substream of the buffer medium. Washing the bioparticles can include applying a first acoustic field gradient to drive the bioparticles from the first medium to the buffer medium. The washed bioparticles can be conveyed from the first fluid pathway into the second fluid pathway while conveying the first medium into a fourth fluid pathway distinct from the second fluid pathway. Trapping the bioparticles can include applying a second acoustic field to establish one or more potential barriers in the second fluid pathway, and accumulating the bioparticles at the one or more potential barriers while the buffer medium flows past the one or more potential barriers. The trapped bioparticles can be periodically extracted from the second fluid pathway and conveyed to the fluid chamber. The method can include combining the bioparticles from the trapping with the labeling particles to form the mixture. The combining can be performed in the second fluid pathway, in a seventh fluid pathway en route from the second fluid pathway to the fluid chamber, or in the fluid chamber. During the incubating, a temperature of the mixture can be controlled to remain within a predetermined range. The incubating can include application of a third acoustic field, to enhance mixing between the labeling particles and the bioparticles, or to enhance mixing among the bioparticles themselves, to reduce dwell time dispersion of the bioparticles in the fluid chamber. Washing the mixture can include driving a third substream of the mixture in contact with and in a same direction as a fourth substream of a second buffer medium. A fourth acoustic field gradient can be applied to the third and fourth substreams, to separate unbound labeling particles from the labeled bioparticles. Extracting the labeled bioparticles can convey the labeled bioparticles and unbound labeling particles into respective fifth and sixth fluid pathways.

In some examples, a fluid sample can be obtained from a first subject prior to washing operation (a), and a suspension of the bioparticles in the first medium can be prepared from the fluid sample. The fluid sample can include first cells which can be cultured in a growth medium to obtain second cells which are the bioparticles. The fluid sample can include antibodies, which can be used as the bioparticles, and which can be separated from other components of the fluid sample. The antibodies can be extracted from the fluid sample and cleaved to obtain antibody fragments, which can be used as the bioparticles. The first subject can be a mammal or a human subject.

In further examples, a sample of a labeling precursor can be obtained prior to incubation operation (c). The labeling precursor can be decomposed to obtain labeling fragments, which can be used as the labeling particles. The labeling fragments can be separated from other decomposition products. The labeling fragments can include antibody fragments or adhesion molecule fragments. Still further, samples of two labeling precursors can be obtained prior to incubation operation (c), and the two labeling precursors can be conjugated to obtain the labeling particles. The labeling particles can be purified. The labeling particles can include antibody fragments or adhesion molecule fragments conjugated with a therapeutic label function or a visualization label function.

In additional examples, following extraction operation (e), a dose can be prepared from the extracted labeled bioparticles. The dose into a second subject. The second subject can be a mammal or a human subject, and can be the first subject or a different subject. The dose can be a diagnostic or a therapeutic.

In some examples, one or more of operations (a)-(e) can be performed within a continuous process. One or more of operations (a)-(e) are performed as a cyclic process. Any of the described methods can be performed as an automated process. Any of the described methods can be performed in a stationary or quasi-stationary apparatus.

In certain examples, the disclosed technologies can be implemented as an apparatus including first through fourth fluid pathways, first through third transducers, and a fluid chamber. The first fluid pathway has first and second inlet ports and first and second outlet ports. The first transducer is configured to produce a first acoustic field gradient in the first fluid pathway. The second fluid pathway has a third inlet port coupled to the second outlet port, and third and fourth outlet ports. The one or more second transducers are configured to (a) produce a first acoustic potential barrier in the second fluid pathway, and (b) relax the first acoustic potential barrier to a second acoustic field barrier. The third fluid pathway has a fourth inlet port coupled to the fourth outlet port, a fifth inlet port, and a fifth outlet port. The fluid chamber has a sixth inlet port coupled to the fifth outlet port, and a sixth outlet port. The fourth fluid pathway has a seventh inlet port coupled to the sixth outlet port, an eighth inlet port, and seventh and eighth outlet ports. The third transducer is configured to produce a third acoustic field gradient in the fourth fluid pathway.

In some examples, the apparatus can also include a substrate upon which two or more of the following are formed: the first fluid pathway, the second fluid pathway, the third fluid pathway, the fluid chamber, or the fourth fluid pathway. The apparatus can include a second substrate upon which one or more of the following are formed: a first acoustic waveguide coupling the first transducer to the first fluid pathway; a second acoustic waveguide coupling the at least one second transducer to the second fluid pathway; or a third acoustic waveguide coupling the third transducer to the fourth fluid pathway.

In certain examples, the disclosed technologies can be implemented as a bioparticle labeling system including any of the above apparatuses, together with: a bioparticle reservoir coupled to the first inlet port; a labeling agent reservoir coupled to the fifth inlet port; first and second wash medium reservoirs coupled to the second and eighth inlet ports; a labeled bioparticle collection chamber coupled to the eighth outlet port; one or more waste collection chambers coupled to the first, third, and seventh outlet ports; drivers coupled to the first, second, and third transducers; and a controller coupled to the drivers.

In varying examples, the bioparticle labeling system can be configured to label cells with radioligands, with magnetic reagents, or with fluorophores, dyes, or proteins. The bioparticle labeling system can be configured to label cells with conjugates of (a) antibodies or antibody fragments and (b) radioisotopes, fluorophores, dyes, drugs, small molecule drugs, nanoparticles, nanotubes or liposomes. The bioparticle labeling system can be configured to label cell membrane components with one or more of: adhesion proteins, receptors, ligands, glycoproteins, or lipids. The bioparticle labeling system can be configured to label cells with one or more of: drugs, inhibitors, small molecule drugs, cytokines, chemokines, nanoparticles, liposomes, biovesicles, or exosomes. The bioparticle labeling system can be configured to label cells with gene components or gene editing reagents.

The foregoing and other objects, features, and advantages of the technology will become more apparent from the following detailed description, which proceeds with reference to the accompanying figures.

### BRIEF DESCRIPTION OF THE DRAWINGS

FIG. 1 is a diagram illustrating a prior art process for cell labeling.

FIG. 2 is a flowchart of a first example method for labeling bioparticles according to the disclosed technologies.

FIG. 3 is a flowchart of a second example method for labeling bioparticles according to the disclosed technologies.

FIG. 4 is a diagram of an example washing apparatus suitable for use according to the disclosed technologies.

FIG. 5 is a diagram of an example trapping apparatus suitable for use according to the disclosed technologies.

FIG. 6A-6E are diagrams illustrating an example trapping operation according to the disclosed technologies.

FIG. 7 is a diagram of an example incubation apparatus suitable for use according to the disclosed technologies.

FIG. 8 is a diagram of an example apparatus and system according to the disclosed technologies.

FIG. 9 is a flowchart illustrating examples of extended methods that can be implemented according to the disclosed technologies.

FIG. 10 is a hybrid diagram illustrating example applications of the disclosed technologies.

FIG. 11A-11B are charts comparing performance in a wash test, including with disclosed technologies.

FIG. 12A-12C are charts comparing performance in labeling tests, including with disclosed technologies.

FIG. 13 illustrates a generalized example of a suitable computing environment in which described embodiments, techniques, and technologies pertaining to beam focusing can be implemented.

### DETAILED DESCRIPTION

### Introduction

Applications abound for labeled cells or other labeled biological particles. In an immunotherapy example, human T cells labeled with a Zr-89 radioligand can be tracked in vivo using PET (positron emission tomography) imaging. In a vaccination therapy example, human dendritic cells labeled with an In-111 radioligand can be tracked in vivo using SPECT (single-photon emission computerized tomography) imaging. In another imaging modality, human dendritic cells labeled with iron oxide nanoparticles or an F-19 perfluorocarbon nanoemulsion can be administered by infusion therapy and tracked by MRI (magnetic resonance imaging). In a diagnostic application, T cells labeled with a CFSE (carboxyfluorescein succinimidyl ester) fluorescent dye can proliferate in vivo, leading to dilution of the dye, which can be monitored using flow cytometry.

In additional applications, antibodies or fragments can be conjugated with a visualization label function and applied to specific cell sites to track migration of infused cells. In one example, a CD8 molecule expressed at the surface of a T cell can be labeled with a conjugate label formed of antibody function Anti-CD8 F(ab′)2 and visualization function Zr-89. The labeled T cells can be administered for immunotherapy and tracked with PET imaging. In another example, a CD45 molecule expressed on the surface of a human dendritic cell can be labeled with a conjugate label formed of antibody function anti-CD45 and a fluorophore visualization function. The labeled dendritic cells can be injected into lymph nodes and monitored in real time using an optical camera, as part of a vaccine therapy.

In further therapeutic applications, a label can be used to enhance effectiveness of a therapeutic agent. In an example, a cell membrane protein can be labeled with an extracellular portion of adhesion molecule, the label increasing delivery of the cell membrane protein to target endothelial cells. In further examples, a label can be used to modify the action of the labeled cell. As an example, regulatory T cells can be incubated with an AKT inhibitor, for use in immunotherapy of acute respiratory distress syndrome. As another example, a CAR label targeting a cancer-specific antigen can be applied to a T cell to enhance cytotoxicity in T cell immunotherapy. As a further example, T cells can be treated with Cas9 and guide RNA targeting a PD-1 gene, leading to production of CRISPR-Cas9 PD-1 knockdown T cells to be used for immunotherapy of cancers.

A cell membrane can provide additional target components for labeling. As an example, fucose sugar units can be attached to cell membrane selectin ligands in a fucosylation process, to enhance adhesion of cells to a target organ or target tissues in a subject, thereby enhancing cell delivery to the target. As another example, a cell membrane lipid layer can be labeled with a lipid mimetic material to form a lipid mimetic luminescent metal complex.

Hitherto, labeling of cells, antibodies, or cell components has been limited by the labor-intensive procedures conventionally used. An exemplary conventional process is described below.

Numerous steps are involved for washing—including centrifugation, aspiration, pipetting, and even simple physical movement of samples from one operation site to another. These steps can involve multiple repetitions of various manual steps, taking a large amount of time, and also tying up laboratory equipment.

In comparison, the disclosed technologies permit a labeling procedure to be performed as a push-button procedure, for example on a microfluidic platform. Once configured for a particular operational sequence and provided with input samples of bioparticles, label particles, suspensions of such particles, or other reagents, the labeling procedure can be performed automatically and without operator supervision. As described further herein, some disclosed embodiments use acoustophoresis as a substitute for washing by centrifugation. Acoustophoretic washing can be performed before and after incubation; the washing stages can be directly coupled to an incubation chamber also implemented on a common microfluidic platform. In further examples, acoustophoretic techniques can be used to enhance mixing or to reduce dwell time dispersion, e.g. in a continuous flow incubation chamber.

Automated labeling can be suitable for each of the various specific applications described above, and many more. The disclosed technologies can be scaled for deployment in large scale trials or studies. Additional advantages and features of the disclosed technologies are brought out through examples described herein.

### A Conventional Process

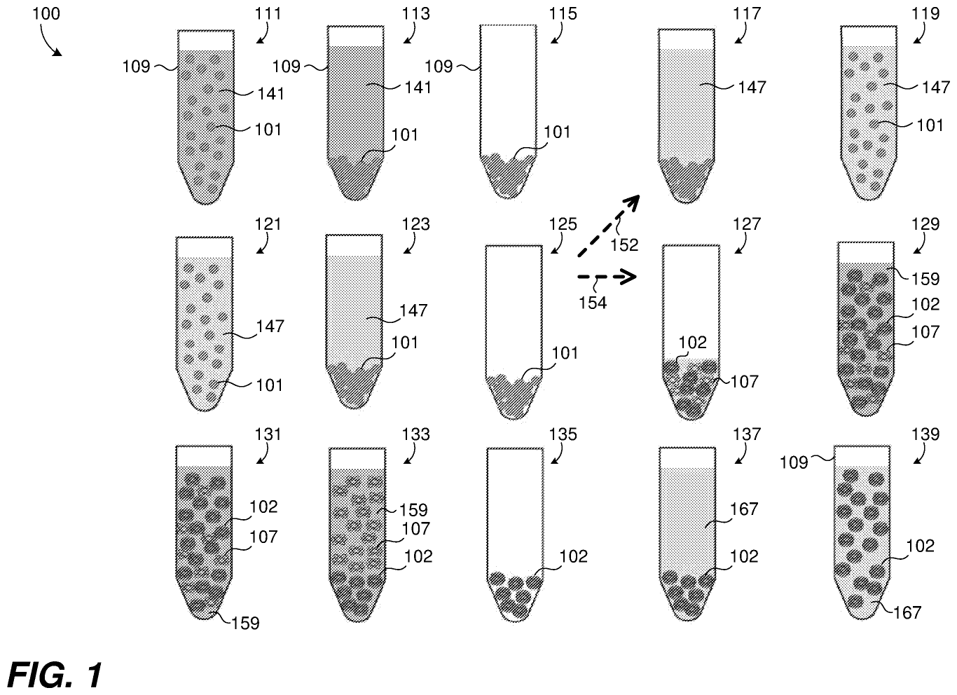

FIG. 1 is a diagram 100 illustrating a prior art process for cell labeling. Illustrations 111-139 represent stages of this process. In examples, the process can be performed in one or more tubes, commonly holding up to about 1-50 mL fluid volume.

Illustrations 111-119 depict a washing process, with a culture medium 141 for cells 101 being replaced by a buffer solution 147. The process starts at 111 with a tube 109 containing a sample of cells 101 suspended in a culture medium 141. As an illustration, a sample of human bone marrow cells 101 can be provided in RPMI 1640 culture medium 141 (Life Technologies, Grand Island, NY). After centrifugation, the cells 101 settle to the bottom of tube 109 as illustrated at 113, under supernatant medium 141. Removal of medium 141 can be performed by needle aspiration or decantation, leaving collected cells 101 in tube 109, still wetted by a residual amount of medium 141, as shown at 115. Then, a buffer medium 147 can be introduced as shown at 117. To illustrate, phosphate buffered saline (PBS) can be used as buffer medium 147. Light agitation, for example using a pipette or in a vortex mixer, can restore the cells 101 from sediment to a suspended state as shown at 119. With culture medium 141 replaced by buffer medium 147, one washing stage is complete.

Illustrations 121-129 depict preparation for incubation, followed by incubation. Illustration 121 can pick up where 119 left off, with cells 101 suspended in buffer 147. The sample can be centrifuged to precipitate cells 101 as shown at 123, followed by removal of supernatant medium 147 to leave cells 101 as residue, as shown at 125. Generally, the sample state at 125 can be similar to 115, however addition and removal of buffer medium 147 at stages 117-125 can remove traces of original culture medium 141 and any components or contaminants therein.

In some examples, the process can continue directly to stage 127, while in other examples additional washing cycles can be performed prior to incubation. That is, the process can return from 125 to 117 as indicated by arrow 152, and the operations associated with stages 117-125 can be repeated one or more times to further remove traces of culture medium 141 in the cell residue at 125. Eventually, with or without additional washing cycles, the process can proceed from 125 to an incubation stage as indicated by arrow 154.

An incubation solution or suspension can be added to the washed cell residue as shown at 127. For example, a radioligand solution (e.g. Zr-89 oxine in PBS, with Tween 80 (MilliporeSigma, St. Louis MO) as a surfactant) can be used as an incubation suspension, with 1:30 dilution of cells by volume in the incubation suspension. The precise amount of radioligand 107 can be calibrated to the count of cells 101. A vortex mixer can be used to enhance mixing of the radioligand 107 with the cells 101. In this illustration, the incubating stage 127 can be maintained for about 15 minutes at about room temperature. Then, culture medium 159 can be added to reduce reactant concentrations and effectively terminate incubation, as shown at 129. To illustrate, the volume of culture medium 159 can be about 10 times the volume of the incubating mixture at 127. The suspension in 129 can include a mix of labeled cells 102 and unbound radioligand particles 107. At 129, the incubation is complete.

Illustrations 131-139 depict another washing stage performed on the incubated and labeled cells 102. Illustration 131 can pick up where 129 left off, with a diluted suspension of primarily labeled cells 102 and excess radioligand particles 107 in a mixed medium which can be mostly culture medium 159. A centrifugation operation can precipitate labeled cells 102 below radioligands 107, medium 159, and other residual constituents, as shown at 133. Removal of supernatant fluids leaves behind a sediment of labeled cells 102 as shown at 135. Generally, the sample state at 135 can be similar to that at 115, except that the unlabeled cells 101 of 115 are now labeled cells 102 in 135. One or more washing cycles can be performed, similar to stages 117-125, to remove any traces of culture medium 159 or incubation suspension. Multiple repetitions of the washing cycle may be required to meet a predetermined specification for removal of unreacted Zr-89 oxine or other labeling particle.

Eventually, with or without these additional washing cycles, the process can continue from 135 to 137, which shows a buffer medium 167 added to the sample tube. Mixing of the contents can produce a suspension of labeled cells 102 in buffer medium 167, as shown at 139. This completes the cell labeling process of FIG. 1.

### Terminology

The usage and meaning of all quoted terms in this section applies throughout this disclosure unless clearly indicated otherwise or repugnant to the context. The terminology below extends to related word forms.

“Acoustic energy” is a form of energy manifested as pressure waves in a material. Acoustic waves can be propagating waves or standing waves, with any standing wave ratio (SWR). A standing wave can have positions where the density of acoustic energy is a local minimum in at least one direction, dubbed “nodes”, and other positions where the acoustic energy is maximum in at least one direction, dubbed “antinodes.” In two or more dimensions, acoustic energy density can be shaped to form a “potential barrier” or a “potential well.” Potential barriers and potential wells are defined from consideration of vibrational energy of an object or material in an acoustic field as potential energy, similar to electrostatic or gravitational potential energy. That is, a particle descending a potential well can experience conversion of its acoustic energy into kinetic energy or another form of energy, while a particle climbing a potential barrier can experience conversion of kinetic energy into acoustic vibrational energy. Accordingly, a potential well can attract a particle, while a potential barrier can repel a particle. Acoustic waves can be bulk acoustic waves (“BAW”), having acoustic energy distributed throughout a volume, or surface acoustic waves (“SAW”), having acoustic energy confined at or near a surface of a material. Accordingly, acoustic energy density can be areal or volumetric energy density, and can have a spatial gradient (dubbed “acoustic energy density gradient” or “acoustic field gradient”). An “acoustic waveguide” having material properties different from surrounding materials can be used to channel the propagation of acoustic waves along a path on a surface or in a volume. Some surface acoustic waveguides can be implemented as interdigitated structures, while some bulk acoustic waveguides can be implemented as solid tubes of arbitrary cross-section embedded in a dissimilar material.

“Acoustophoresis” refers to a process that controls motion of particles or other objects with acoustic energy. An “acoustic field” is a distribution of acoustic energy through a region. Because the acoustic energy density can be non-uniform, movable objects in an acoustic field can have different vibrational energies at different positions in the acoustic field: the spatial derivative of this energy (“gradient”) can be experienced as a force on the movable object. The energies and forces can be dependent on the object material and can act differentially on two objects of different materials. In some examples, a heavy object (e.g. a bioparticle) can be present at a position of high acoustic energy density, while a lighter material (e.g. an equal volume of a medium) can be present at an adjacent position of lower acoustic energy density. Exchange of position between the heavy bioparticle and the lighter medium can take place because it can lead to a net lowering of acoustic energy and can therefore be energetically favored. Thus, acoustic waves or gradients in acoustic energy density can exert forces on particles or on a medium, which can cause movement of the particles or the medium. In other examples, an arrangement of objects and material in an acoustic field can be at an energy minimum, and motion across acoustic field gradients can be inhibited. That is, acoustophoresis is not restricted to causing a motion but can also be exhibited as preventing a motion from occurring. Examples of the former can include causing particles to move from one substream to another within a fluid pathway. Examples of the latter can include trapping particles at a trapping site or keeping two adjacent substreams flowing parallel without mixing.

An “adhesion molecule” is a protein that can control binding or other interactions between two proximate cells, between a cell and a label, or between a cell and an extra-cellular matrix. Decomposition of an adhesion molecule can generate two or more “adhesion molecule fragments.” Adhesion molecules or their fragments are of interest as label particles either by themselves or conjugated with another label function such as a drug or fluorophore. That is, conjugation with an adhesion molecule or fragment can facilitate binding of a label function to a cell or other bioparticle.

An “antibody” is a protein able to counter an antigen. Decomposition of an antibody can generate two or more “antibody fragments.” While antibodies are commonly produced in vivo, this is not a requirement. Antibodies produced in vivo can also be cultured in vitro, and other synthetic antibodies can be manufactured or produced fully in vitro. In some disclosed examples, antibodies or their fragments can be used as bioparticles, while in other examples antibodies or their fragments, can be used as or in labeling particles. Useful antibody fragments include, without limitation, F(ab′)₂ or minibodies.

A “bioparticle” is an object with defined boundaries that is or contains one or more organic molecules and is capable of playing a role in a biological process. Because of their role in biological processes, it can be valuable to tag bioparticles with labels that can make them visible to a diagnostic, so that the tagged bioparticles can be tracked either in vivo or in vitro. In other embodiments, labels can also be applied to enhance or modify a biological function. Bioparticles of interest in this disclosure include cells, cell components, antibodies, and antibody fragments. Some bioparticles of interest in this disclosure can be extracted or cultured from a sample obtained from a plant or animal subject. However, bioparticles are not so limited: some non-biological or “man-made”compounds such as drugs or proteins can also be bioparticles. A bioparticle can be a stand-alone object, can be enclosed within a droplet or nanotube, or can be attached to a carrier such as a microsphere or bead. In further examples, the bioparticle can be a passive carrier for a label such as a drug molecule, where the desired functional activity is performed by the label rather than by the underlying bioparticle. Still further, labeling a cell with a drug molecule can modify the cell into a treated cell, so that treated cells can be delivered to a subject or used in a study.

A “cell” is a biological unit having proteins and nucleic acids in a fluid enclosed within a membrane (“cell membrane”). A “viable” cell is a cell that is capable of performing one or more of its biological functions according to a predetermined test. A “dead” cell is incapable of performing its biological functions. “Cell viability” is the proportion of viable cells within a sample of cells. The term “cell component” refers to DNA, RNA, mitochondria, or lysosomes, any of which can be a target for labeling within a cell. Some cell components such as DNA or RNA can also be bioparticles suitable for labeling outside a host cell. On a cell membrane, various components such as proteins, ligands, or lipids can be labeled. Cell membrane components suitable for labeling are not limited to proteins, and labels other than proteins can be applied to cell membrane components.

The terms “chamber,” “channel,” and “pipe” refer to generally enclosed receptacles within which a fluid can flow or can be stored. For clarity of illustration, the term chamber can be used for receptacles having the ratio of two longest interior dimensions not exceeding 10:1, while the term pipe can be used for receptacles whose two longest interior dimensions have a ratio exceeding 10:1. However, such a distinction is not limiting, and the terms chamber, channel, and pipe can be used interchangeably. The presence of one or more ports does not preclude a structure from being a chamber, channel, or pipe.

“Cleave” refers to an operation of breaking one or more targeted chemical bonds in a molecule. While cleavage can often result in two fragments, this is not a requirement. In some cyclic molecular structures, cleavage of a single bond can be insufficient to fragment the molecule, while in other instances, cleavage can result in three or more fragments. In some examples, cleavage of a long chain molecule at two nearby sites can result in “snipping” of the portion between the two sites.

A “controller” is an electronic apparatus having outputs interfaced to one or more actuators or other devices which make physical changes in the device's physical environment. For example, a controller can operate a pump, a valve, a driver for an acoustic transducer, a pneumatic actuator, a heating and/or cooling subsystem, or an optical diagnostic. Some controllers can monitor signals received from sensors of physical parameters in the physical environment. Controller output can be at least partially based on such signals, however this is not a requirement, and other controllers can be operated without sensors. Some controllers can be implemented as computing systems with one or more hardware processors. However, this is not a requirement, and other controllers can be implemented with discrete logical or analog circuits.

As a verb or qualifying adjective, the term “culture” refers to maintaining cells in a viable state. While cells can grow in size or number when cultured, this is not a requirement. As an example, cells can be placed in a culture medium with suitable pH and at a suitable temperature. A “culture medium” is a nutrient broth containing proteins, sugars, and/or other nutrients to sustain cell metabolism. A variety of culture media can be used, such as RPMI, or a protein containing solution like PBS serum.

A “cyclic process” is a process in which a sequence of distinct states recurs. Some cyclic processes can be periodic, but this is not a requirement. In other examples, states of a cyclic process can occur at varying intervals. An example of a cyclic process is trapping of bioparticles in a stream, which can alternate between an accumulation state, in which entering bioparticles are collected at a trapping site, and a discharge state, in which accumulated bioparticles are released. This example of a cyclic process can have a continuous input stream and batch output. Other cyclic processes can have batch inputs and batch outputs. To illustrate, some example incubation chambers can receive a predetermined number (e.g. one or more) of input batches of trapped bioparticles, which can then be incubated together with no further inputs, and the entire amount of incubated bioparticles can then be released together as an output batch. (Other examples of incubation chambers can operate in continuous mode, where additional input batches of trapped bioparticles continue to be introduced from an inlet port even as incubation of earlier input batches is ongoing.)

The term “decompose” refers to any process for separating an object into constituent parts. Examples of decomposition include cell lysis or molecule cleavage.

A “dose” is a predetermined quantity of a substance to be administered to a subject for diagnosis or treatment of a biological condition or process. Exemplary diagnostic substances can include cells or other bioparticles labeled with fluorophores, radioligands, or MRI contrast agents. Exemplary therapeutic substances can include cells or other bioparticles labeled with drugs, antibodies, or adhesion molecules.

As a verb, “drive” refers to an operation in which an object or material is moved from one position to another. For example, a fluid stream can be driven through a fluid pathway, or a bioparticle can be driven from one substream to another.

A “driver” is a device that provides energy to a material object or another device. Some drivers described herein provide electrical energy to piezoelectric transducers to generate acoustic waves or fields. Other drivers can be pumps that drive fluids (including suspensions) through a microfluidic apparatus, or actuators for valves. Further drivers can be energy sources for heating and/or cooling subsystems, or for lasers or other diagnostic instrumentation.

A “drug” is a substance that controls, modifies, or treats a biological process or condition when administered to a subject.

A “dye” is a substance having a color. In some examples, dyes can be used to label bioparticles to render them optically distinguishable from their immediate surroundings. The color of a dye can appear when illuminated by light (infrared, visible, or ultraviolet) or can be expressed through fluorescence. Some DNA binding dyes are fluorescent materials attachable to nucleic acids. Some viability dyes can be selectively attached to living (viable) or dead cells, e.g. as an aid to determining cell viability. Some ion indicator dyes are fluorescent dyes having affinity for ions such as Ca²⁺, K⁺, or Na⁺.

An “extracellular vesicle” (or “biovesicle”) is a class of bioparticle distinct from cells and can include exosomes, microvesicles, or apoptic bodies. Like other bioparticles, extracellular vesicles can be candidates for labeling. Generally, descriptions of operations performed with cells or other bioparticles herein can also be performed with extracellular vesicles. Some extracellular vesicles can be synthetic. In other examples, an extracellular vesicle can be a labeling particle applied to a cell.

A “fluid” is a homogeneous or heterogeneous liquid. In some examples, a fluid can be a homogeneous, substantially pure medium, such as a wash medium introduced as a substream into a washing apparatus. In other examples, a fluid can be a heterogenous liquid having particles, droplets, particle carriers, reaction products or byproducts or contaminants suspended or carried by a stationary or flowing medium. In some examples, a fluid can flow in a pathway, or can flow or be stored in a chamber. A fluid “pathway” is a defined path through which one or more streams or substreams of fluid can flow. A pathway can be a closed channel defined by interior surfaces of a microfluidic platform, but this is not a requirement, and in other embodiments open channels or uncovered fluid chambers can also be used.

A “fluorophore” is a chemical compound that can emit light of one wavelength upon excitation by light of another wavelength. This emission of light is dubbed “fluorescence.” “Light” can refer to electromagnetic radiation in the infrared, visible, or ultraviolet portions of the spectrum, and is not limited to visible light.

“Incubation” is an operation of maintaining one or more objects or reaction components in conditions favorable for a chemical or biological process. In some examples, a mix of bioparticles and labeling particles in a medium is incubated for a predetermined period of time to enable the labeling particles to be bound to (or, bound within) the bioparticles. Incubation can be done under temperature controlled conditions, but this is not a requirement. Incubation can be performed as a continuous process or as a batch process. In the latter case, two or more incubation chambers can be used in tandem. While one chamber is incubating, incoming reaction components can be loaded into another chamber for a subsequent incubation batch.

An “inhibitor” is a substance or particle which slows down or blocks a biological process.

As a noun, the term “label” refers to a substance that can be attached to another substance, for example to assist with diagnosis, monitoring, or treatment of a biological process or condition. To illustrate, radioligand or magnetic labels can be used to visualize an underlying labeled bioparticle using PET or MRI respectively. A label can have one or more functions, including visualization, treatment, or selective attachment. To illustrate, a fluorophore can provide a visualization function, a drug can provide a treatment function, or an adhesion molecule can provide a selective attachment function. Two or more label functions can be combined through conjugating components having the respective functions. A label can be bound to a bioparticle (a “labeled bioparticle”) or can be unbound (e.g. prior to an incubation operation, or as excess left over after incubation). As a verb, the term “label” refers to a procedure for attaching a label to a bioparticle.

A “liposome” is a lipid membrane enclosing a water droplet or other objects.

The term “magnetic” refers to materials or particles which can be visualized through magnetic resonance imaging (MRI). Paramagnetic iron oxide nanoparticles are an example.

A “medium” is a liquid in which other particles or objects can be stored or carried. Media can be variously described according to their role in a labeling process, such as a buffer medium; a culture, growth, or incubation medium; or a wash medium.

“Microfluidics” refers to control and manipulation of fluids in channels or chambers having two or more dimensions in a range 1-1000 μm. Microfluidic apparatus can be implemented with channels formed in a substrate, and can include auxiliary devices such as valves, pumps, acoustic transducers, heating and/or cooling subsystems, or diagnostic sensors or instruments. A microfluidic apparatus can have multiple sections configured to perform respective operations on fluid samples, with the various sections coupled together on a single substrate. “Nanofluidics” is similar to microfluidics, with at least one dimension of a channel or chamber being in a range 1-1000 nm. “Millifluidics” is similar to microfluidics, with at least two dimensions of a channel or chamber being at most 10 mm, and at least one dimension in a range 1-10 mm. Millifluidics apparatus can also include channels or chambers having one dimension of at most 5 mm and another dimension in a range 2-50 mm. While some apparatuses are described herein as microfluidic for convenience of description, this is not limiting, and nanofluidic or millifluidic embodiments are included within such description. Particularly, a millifluidic incubation chamber can be integrated with a microfluidic washing apparatus in some examples.

A “mixture” is an interspersed combination of two or more distinct types of objects or materials. Some mixtures described herein incorporate two distinct particle species in a medium.

A “nanoparticle” is a solid object or structure having two or more dimensions between 1 and 500 nanometers. Some nanoparticles can be “nanotubes,” which are cylindrical shells, having cross-section dimensions up to 500 nm, about an internal volume. A nanoemulsion is a dispersion of droplets of a given substance within a medium, with the droplets having characteristic size between 1 and 500 nanometers.

A “port” is an aperture through which a fluid can enter or exit an apparatus. A fluid can enter the apparatus at an “inlet port,” or can exit at an “outlet port.” An inlet port of one apparatus or section can be coupled to an output port of another apparatus or section. A port can have a physically distinguishable structure, but this is not a requirement. For example, in an integrated microfluidic apparatus, a cross-section of a uniform fluid channel can be both an outlet port of a preceding section and an inlet port of a succeeding section. Occasionally, under conditions of flow reversal, inlet and outlet ports can exchange roles.

A “process” is an operation or sequence of operations. Some operation of interest herein are automated, batch, continuous, or cyclic. Once set up, an automated process can be performed entirely by a controller, with no manual operations required. Here, manual operations include robotic operations, e.g. for moving a sample in its container from one location to another. A “continuous process” is a process in which inputs are received without interruption, the inputs are processed as received to generate outputs, and the outputs are delivered without interruption. In contrast to a continuous process, a “batch process” can operate on one batch of inputs at a time.

A “reagent” is a material used or intended for use in a chemical reaction. Some reagents described herein can undergo binding to a target bioparticle, or to a conjugating species.

A “reservoir” is a macroscopic supply of a fluid having mass at least 1 μg. A reservoir can be enclosed within a chamber.

A “sample” is a quantity of material on which an operation is performed. Examples of samples can include bioparticles, labeling particles, or suspensions of such particles or bioparticles, or mixtures of multiple species e.g. in an incubation chamber.

As a verb, “separate” refers to an operation of physically moving one component of a heterogeneous material or sample apart from another distinct component. In some examples, separation can effect purification of a separated component. To illustrate, separation of a desired fragment from cell lysis can remove other byproducts and can purify the desired fragment.

A “small molecule drug” (or simply “small molecule”) is a molecule having a molecular weight below 900 daltons and a demonstrated capacity to regulate a biological process. Thus, proteins and nucleic acids (RNA, DNA, RNA) are generally excluded from small molecules. Because of its small molecular weight, a small molecule drug can enter cells easily. Once inside a cell, a small molecule drug can affect other molecules such as proteins, and in some instances can cause cancer cells to die. In some examples, a drug molecule can be attached as a label to a bioparticle to assist with delivery of the drug molecule. In other examples, a drug molecule can treat the bioparticle to which it is attached, allowing treated bioparticles to be efficiently produced ex vivo, for subsequent delivery to a subject. Such a technique can provide targeted therapy and can avoid side effects.

A “stationary” apparatus is an apparatus having no moving parts, i.e. no parts that can move relative to one another in normal operation. An apparatus being stationary does not preclude its use with fluids that move through the apparatus. A “quasi-stationary” apparatus can have discrete movable valves to control fluid motion in an otherwise stationary apparatus.

A “stream” is a generally linear flow of a fluid along a path. A stream can change direction according to follow guiding walls or in response to acoustophoretic forces. A stream can be composed of multiple substreams flowing in parallel along the path. Substreams can retain their identity as the stream flows or can mix. In some examples, acoustophoresis can be used to inhibit mixing of two substreams, while in other examples acoustophoresis can be used to enhance mixing. A stream or substream can carry objects (such as bioparticles, labeling particles, droplets, or particle carriers) as it flows.

A “subject” can be a living human, other mammal, other animal, or plant from whom or from which a cell or fluid sample is obtained, or to whom or to which a dose is administered.

A “suspension” is a heterogeneous material composition having solid particles or bioparticles supported against gravity in a fluid (often liquid) medium. A suspension can contain multiple species of particles or media. In some examples, the solid particles or bioparticles can be encapsulated in individual droplets of a second medium immiscible with a primary medium of the suspension. For example, bioparticles can be encapsulated in oil or lipid droplets, with the droplets (including bioparticles) suspended in an aqueous medium.

A “transducer” is a device that converts one form of energy into another. Some transducers of interest in this disclosure are acoustic transducers, which convert electrical energy into acoustic energy. Some acoustic transducers can be piezoelectric devices.

As a verb, “trap” refers to an operation of collecting particles in a flow at a site known as a “trapping site.” As a noun, “trap” can refer to the trapping site or to an apparatus in which trapping can be performed.

“Wash” refers to an operation of exchanging a first medium for a second medium in a particulate suspension.

### First Example Labeling Method

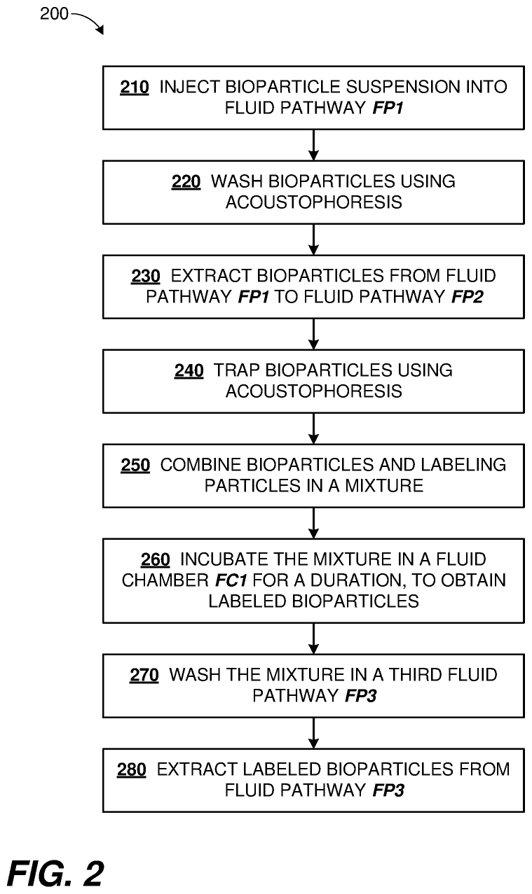

FIG. 2 is a flowchart 200 of a first example method for labeling bioparticles according to the disclosed technologies. In this method, acoustophoresis is used in several steps of an integrated process to apply labels to bioparticles. The method is carried out in a series of fluid pathways and a fluid chamber, and can suitably be implemented on a microfluidic platform.

At process block 210, a bioparticle suspension can be injected into a fluid pathway FP1. To illustrate, the bioparticle suspension can be a suspension of T cells in a growth medium or other suspension medium. The bioparticle suspension can propagate along fluid pathway FP1. In fluid pathway FP1, the bioparticle suspension can be a first substream propagating alongside and in contact with a second substream. The second substream can be a buffer medium such as PBS.

At process block 220, the bioparticles can be washed using acoustophoresis. To illustrate, an acoustic field gradient in fluid pathway FP1 can drive the bioparticles transversely from the first substream to the second substream as they flow along fluid pathway FP1 in a region having an acoustic field.

After the bioparticles have been washed from the suspension medium to the buffer medium, the bioparticles can be extracted from fluid pathway FP1 into another fluid pathway FP2 at process block 230. To illustrate, the bioparticles can by conveyed from pathway FP1 to pathway FP2 along with a portion of the buffer medium, while the suspension medium and another portion of the buffer medium can be conveyed from pathway FP1 along a different pathway. Thus, in blocks 210, 220, 230, the bioparticles can enter fluid pathway FP1 with a suspension medium and can exit the fluid pathway FP2 with a buffer medium, completing a washing stage of the instant method.

In fluid pathway FP2, the bioparticles can be trapped using acoustophoresis at process block 240. To illustrate, an acoustic field in fluid pathway FP2 can form a potential barrier at which the bioparticles can be trapped, even as the washing medium continues to flow past. That is, at process block 240, an incoming flow of washed bioparticles can be collected at a trapping site.

Thereafter, the trapped bioparticles can be combined with labeling particles to form a mixture at process block 250. As an illustration, the labeling particles can be Zr89-oxine, a radioligand.

At process block 260, the mixture can be incubated in a fluid chamber FC1 for a predetermined duration, to develop labeled bioparticles. As an illustration, a radioligand labeling particle can penetrate a bioparticle cell wall, and then become bound to a protein inside the cell. While cell penetration can occur quickly, e.g. in under a minute, protein binding can take significantly longer: on the order of 15 minutes in some examples. In varying examples, fluid chamber FC1 can be operated as a batch mode incubator, or as a first-in-first-out (FIFO) fluid channel with wide cross-section. A FIFO fluid chamber can be operated with continuous flow or with peristaltic flow, advancing each time a batch of trapped bioparticles is delivered to fluid chamber FC1. Dispersion of incubation time can be mitigated by various techniques as described herein. Fluid chamber FC1 can be equipped with temperature control, to maintain contents of chamber FC1 within a predetermined temperature range, or diagnostics, as also described herein. Whether operated as a FIFO channel or a batch-mode reactor, unlabeled bioparticles entering the chamber FC1 can emerge as labeled bioparticles. The incubated mixture can also contain unbound (excess) labeling particles.

At process block 270, the mixture can be washed in fluid pathway FP3. Particularly, the labeled bioparticles can be washed from a first medium (the mixture in which incubation was performed) into a second buffer medium, in a similar manner as described in context of block 220 herein. Under influence of an acoustic field gradient, the labeled particles can be driven from a substream containing the incubated mixture into another substream containing the second buffer medium, causing labeled bioparticles to be separated from unbound labeling particles. Then, the labeled particles can be extracted along with a portion of the second buffer medium at block 280, while unlabeled bioparticles can be retained in the first medium and can be directed out from pathway FP3 along a different pathway.

Numerous variations and extensions of this method can be implemented within the scope of the disclosed technologies, some of which have been described above for purpose of illustration. The method can be applied to many different classes of bioparticles and labeling particles. For the bioparticles, exemplary alternatives to cells include cell components, cell membrane components, antibodies, antibody fragments, biovesicles, or proteins can be used. Some cells of interest can include T cells, regulatory T cells, or dendritic cells. Cellular bioparticles can be viable cells or dead cells. Cellular bioparticles can be mammalian or human cells. Protein bioparticles can include cell membrane proteins or other cellular proteins. Bioparticles can be self-contained, such as cells enclosed within their respective cell membranes, or can be attached to carrier particles. Various carrier particles including cells, microspheres, nanostructures (e.g. nanoparticles or nanotubes), beads, or fluid droplets can be used to carry or encapsulate bioparticles.

Turning to the labeling particles, exemplary alternatives to radioligands include adhesion molecules, adhesion molecule fragments, antibodies, antibody fragments, biotin esters, drugs, dyes, fluorophores, glycoproteins, ligands, lipids, liposomes, inorganic nanoparticles, nanotubes, magnetic labels, proteins, receptors, or small molecules. Particular examples of labeling particles include carboxyfluorescein succinimidyl ester (CFSE), other fluorescent dyes, fluorophore conjugated antibodies, fluorescent expression proteins, iron oxide nanoparticles, F-19 perfluorocarbon nanoemulsions, DNA binding dyes, viability dyes, ion indicator dyes, Zr-89 oxine, In-111 oxine, or a Zr-89 conjugated antibodies. Additional examples of labeling particles include fucose sugar units or lipid mimetic materials.

In further examples, the trapped bioparticles can be periodically extracted from fluid pathway FP2 and conveyed to incubation chamber FC1. In varying examples, combining the labeling particles with the extracted bioparticles can be performed in fluid chamber FC1, en route from pathway FP2 to chamber FC1, or in pathway FP2.

Other features of incubation chamber FC1 can include acoustic fields configured to develop relative motion between labeling particles and bioparticles, to enhance mixing between the two particle types. In varying examples, constrictions or diagonal ridges can be implemented within chamber FC1 to cause mixing among the bioparticles and reduce dispersion of dwell time in flowing examples of chamber FC1. Constrictions, diagonal ridges, or other topographical features of chamber FC1 can be implemented through physical structures that can mechanically guide fluid or particle flow, or through acoustic fields that can cause particle motion or mixing by acoustophoresis.

In some examples, all process blocks of FIG. 2 can be performed together as an automated process, such as on a microfluidic platform. In some examples, some or all of the process blocks of FIG. 2 can be performed as a continuous process, and in further examples some or all of the process blocks of FIG. 2 can be performed cyclically. In particular examples, blocks 210-230 can be performed continuously (over the duration taken to process a given batch or supply of the bioparticle suspension), while blocks 240-280 can be performed cyclically in batches. In other examples, blocks 240-250 can be performed cyclically in batches, while the remaining blocks 210-230 and 260-280 can be performed continuously. As described further herein, trapping can lead to an increase in bioparticle concentration. Thus, in further variations, a technique to increase concentration in a continuous mode can be substituted for block 240, following which block 250 can also be operated continuously.

### Second Example Labeling Method

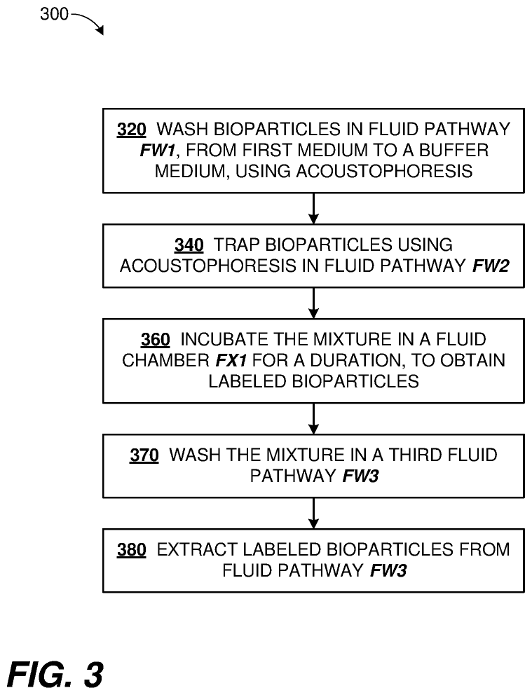

FIG. 3 is a flowchart 300 of a second example method for labeling bioparticles according to the disclosed technologies. As in the previous method, acoustophoresis is used in several steps of an integrated process to apply labels to bioparticles. The method is carried out in a series of fluid pathways and a fluid chamber, and can suitably be implemented on a microfluidic platform.

At process block 320, the bioparticles can be washed from a first medium to a buffer medium using acoustophoresis. To illustrate, an acoustic field gradient in fluid pathway FW1 can drive the bioparticles transversely from a first substream of the first medium to a second substream of the buffer medium, as the substreams flow along fluid pathway FW1 in a region having an acoustic field.

At process block 340, the washed bioparticles can be trapped in fluid pathway FW2 using acoustophoresis. To illustrate, an acoustic field in fluid pathway FW2 can form a potential barrier at which the bioparticles can be trapped, even as the washing medium continues to flow past. That is, at process block 340, an incoming flow of washed bioparticles can be collected at a trapping site.

At process block 360, a mixture of labeling particles and bioparticles extracted from the trapping site can be incubated in a fluid chamber FX1 for a predetermined duration, to develop labeled bioparticles. Unlabeled bioparticles entering chamber FX1 can emerge as labeled bioparticles. The incubated mixture can also contain unbound (excess) labeling particles.

At process block 370, the mixture can be washed in fluid pathway FW3. Particularly, the labeled bioparticles can be washed from a second medium (the mixture in which incubation was performed) into a second buffer medium, in a similar manner as described in context of block 220 herein. Under influence of an acoustic field gradient, the labeled particles can be driven from a substream containing the incubated mixture into another substream containing the second buffer medium, causing labeled bioparticles to be separated from unbound labeling particles. Then, the labeled particles can be extracted along with a portion of the second buffer medium at block 280, while unlabeled bioparticles can be retained in the second medium and can be directed out from pathway FW3 along a different pathway.

Numerous variations and extensions of the second labeling method can be implemented within the scope of the disclosed technologies. In some examples, an operation similar to that of block 210 can be performed prior to block 320, to inject bioparticles in a suspension into fluid pathway FW1. In some examples, an operation similar to that of block 230 can be performed between blocks 320, 340, to extract washed bioparticles from pathway FW1 to pathway FW2. Numerous other variations are described in context of FIG. 2 or elsewhere herein.

### Example Washing Apparatus

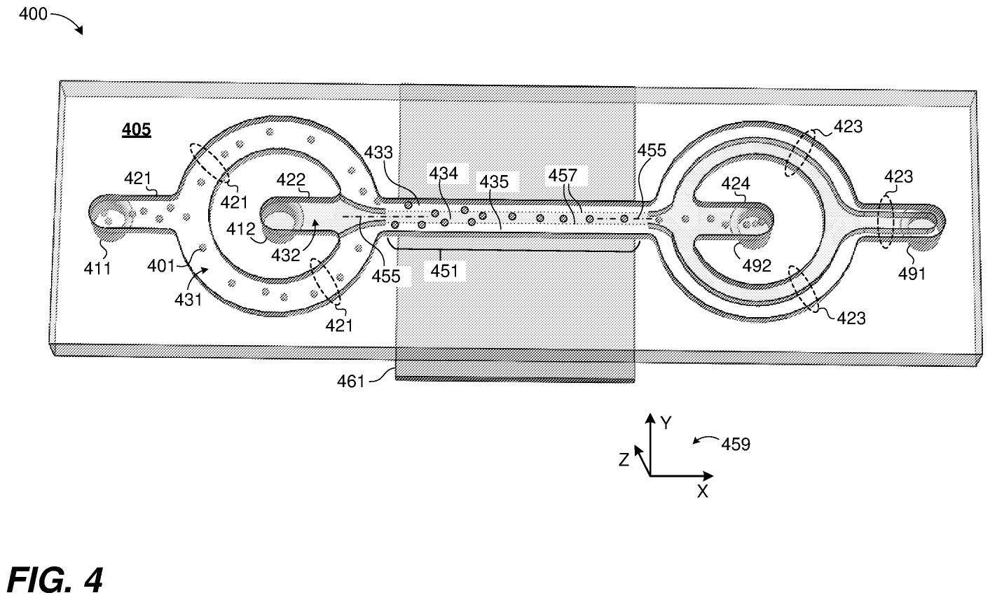

FIG. 4 is a diagram of an example washing apparatus 400. In apparatus 400, acoustophoresis is used to drive particles from one substream to another. Illustrated apparatus 400 is a microfluidic device, however this is not a requirement, and other fluid handling technologies can also be used for acoustophoretic washing according to the disclosed technologies. For clarity of illustration, FIG. 4 shows fluids and particles in apparatus 400, however these are not part of apparatus 400 per se.

Apparatus 400 can have inlet ports 411, 412 and outlet ports 491, 492. Respective fluid channels 421, 422 can couple inlet ports 411, 412 to fluid pathway 451, while fluid channels 423, 424 can couple fluid pathway 451 to outlet ports 491, 492. Channels 421-424 and fluid pathway 451 can be formed in a substrate 405. In operation, inlet port 411 can provide particles 401 in a source medium 431 via fluid channel 421 to fluid pathway 451, while inlet port 412 can provide a wash medium 432 via fluid channel 422 to fluid pathway 451. Within fluid pathway 451, incoming media 431, 432 can form distinct substreams 433-435 as shown. Dotted lines 457 represent boundaries between substreams 433-435.

Acoustic transducer 461 can impose standing acoustic waves over the region of fluid pathway 451. In the illustration, a node line of the acoustic field can run along a longitudinal centerline 455 of fluid pathway 451. A node line corresponds to the lowest energy density (zero) of a standing wave acoustic field and can attract particles toward the center where wash medium 432 flows in substream 434. That is, transverse gradients of the acoustic energy density can drive particles 401 from off-axis regions having higher energy density to on-axis where acoustic energy density is low.

Similarly, differential acoustic forces on wash medium 432 and source medium 431 can maintain the relative positions of substreams 433-435 as the substreams flow towards outlet channels 423, 424. In some examples, a small density difference between source medium 431 and wash medium 432 can be sufficient to maintain separation of fluid substreams 433-435. To illustrate, a PBS source medium 431 can have a density of 1.006, while a wash medium can be a heavy-PBS blend with density about 1.009. An exemplary heavy PBS blend can be formed as a 20:1 mix of PBS and a lymphocyte separation medium (density 1.077). Thus, the higher density wash medium 432 can experience greater acoustic force towards the acoustic node along centerline 455 of pathway 451, relegating lighter source medium 431 to antinode regions away from centerline 455.

As shown, substreams 431, 432 propagate along pathway 451 towards outlets 491, 492. Exiting fluid pathway 451, the flow of substreams 433-435 can be distributed among channels 423, 424 based on relative impedances of the channels, fluid momentum, and the precise physical configuration of the transition between pathway 451 and channels 423, 424. As illustrated, particles 401 and a central portion of substream 434 can exit through channel 424 to outlet 492, while the outer portions of substream 434 and all of substreams 433, 435 (including source medium 431) can exit through channel 423 to outlet 491.

As described, particles 401 can enter apparatus 400 with source medium 431 and can exit with wash medium 432, and washing can be accomplished in a single operation. The microfluidic platform 400 can be integrated with other operational units as described herein, to implement a complete and compact labeling apparatus for an automated labeling process.

Numerous variations and extensions of apparatus 400 can be implemented within the scope of the disclosed technologies. An acoustophoretic washing apparatus can be used in a labeling application for washing: bioparticles, label particles, other species (e.g. adhesion molecules), or particle assemblies (e.g. labeling particle in a droplet, or bioparticle on a microsphere carrier). In varying examples, and dependent on the particular particle species involved, or their sizes or volumes, a washing apparatus can be implemented using nanofluidic or millifluidic structures. In other variants, a similar structure can be operated with particle suspension injected into inlet port 412 and wash medium 432 injected through port 411. In such an example, heavier particles can remain fixed on centerline 455 through pathway 451, while wash medium 432 can exchange places with lighter source medium 431. In some examples, blocks 210, 220, 230 of FIG. 2 or block 320 of FIG. 3 can be performed using an apparatus similar to 400, with pathway 451 for FP1 or FW1. In some examples, blocks 270, 280 of FIG. 2 or block 370, 380 of FIG. 3 can be performed using an apparatus similar to 400, with pathway 451 for FP3 or FW3. Multiple washing stages can be coupled in series. In some tests, two stages of acoustophoretic washing have been found to be as effective as four stages of conventional washing each using centrifugation and aspiration, e.g. for separation of labeled bioparticles from unbound labeling particles in an incoming suspension.

Acoustic transducer 461 has been described as setting up an acoustic energy density with variations in the Y direction shown at axes 459. In further examples, the same transducer 461 or another transducer can be used to create additional acoustic energy density variations in the Z direction shown, with a node at a midplane half-way between top and bottom faces of plate 405, thereby keeping particles 401 away from top and bottom surfaces of pathway 451.

The illustrated symmetric planar configuration of three substreams 433-435 is only an example. In other examples, substream 435 can be omitted and washing can be performed using just two substreams, with corresponding changes to the acoustic field pattern in pathway 451 and the physical design of inlet and outlet channels 421-424. With two substreams, the acoustic field node can be offset from centerline 455 so as to remain at a centerline of substream 434.

The rate RP at which washed particles 401 exit through outlet 492 can be proportional or equal to the rate at which particles 401 enter apparatus 400 at inlet 411, and can be independent of fluid velocity in substream 434. The volume flow rate RM of wash medium 432 exiting outlet 492 can be dependent on or proportional to fluid velocity in substream 434. Accordingly, the concentration of washed particles (which is proportional to RP/RM) exiting outlet 492 can be controlled by varying one or both of RP or RM. In some examples, the fluid velocity in substream 434 can be chosen to achieve a target concentration value of washed particles in the substream exiting at outlet 492. Increasing particle concentration by flow rate control can be used additionally or alternatively to trapping described herein.

### Example Trapping Apparatus

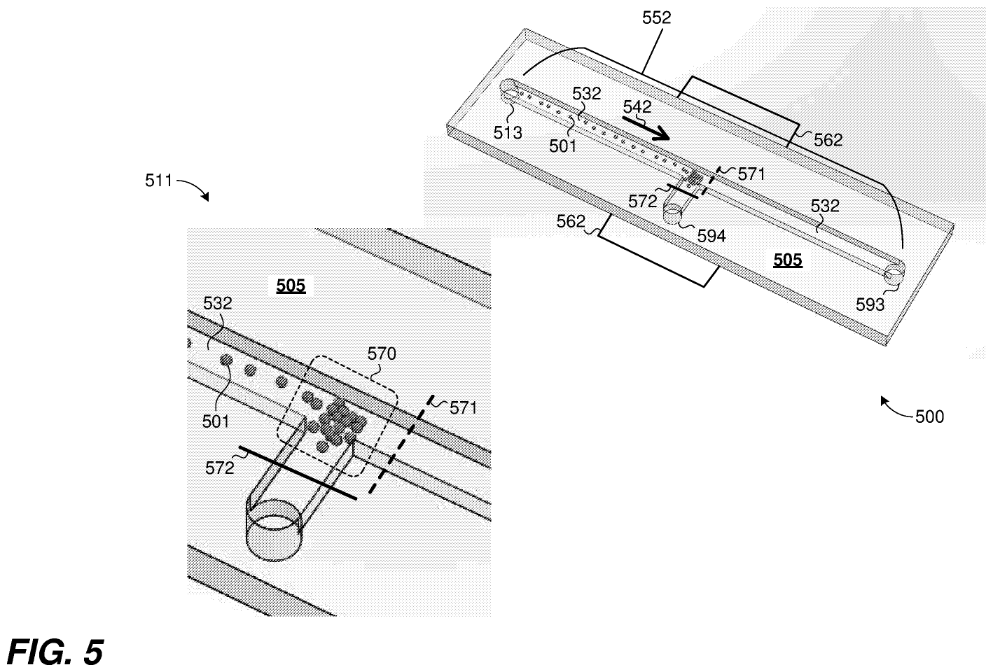

FIG. 5 is a diagram of an example trapping apparatus 500, with inset 511 providing additional detail. Illustrated apparatus 500 is a microfluidic device fabricated in a substrate 505, however this is not a requirement, and other fluid handling technologies can also be used for acoustophoretic trapping according to the disclosed technologies. For clarity of illustration, FIG. 5 shows fluids and particles in apparatus 500, however these are not part of apparatus 500 per se.

In apparatus 500, fluid pathway 552 couples inlet port 513 to outlet port 593, with an additional outlet port 594 for emptying the trap as described further herein. Generally, inlet port 513 can introduce a suspension of particles 501 in medium 532 into pathway 552, flowing in a direction shown by arrow 542.

In a first operating configuration, one or more acoustic transducers 562 can create an acoustic energy field having potential barriers 571, 572. In this configuration, barrier 572 (solid line) can block both medium 532 and particles 501 from egress at outlet 594, while barrier 571 (dashed line) can block particles 501 but can be permeable to medium 532. Thereby, particles can be blocked from proceeding toward outlets 593, 594 respectively, even as medium 532 continues to flow to outlet 593.

In a second operating configuration, barrier 571 can be maintained intact while barrier 572 can be lowered, allowing particles 501 and medium 532 to discharge through side outlet 594.

Numerous variations and extensions of apparatus 500 can be implemented within the scope of the disclosed technologies. A trapping apparatus can be used in a labeling application for trapping: bioparticles, labeling particles, other species, or particle assemblies. In varying examples, and dependent on the particular particle species involved, or their sizes or volumes, a washing apparatus can be implemented using nanofluidic or millifluidic structures. In some examples, a same acoustic transducer can configured to generate both barriers 571, 572. That is, a single transducer can be excited to generate different acoustic field patterns so as to switch between first and second configurations. In other examples, separate transducers can be implemented for barriers 571, 572 respectively. In further examples, acoustophoretic barriers can be supplemented or replaced by mechanical valves (including microfluidic membrane valves). Particularly, barrier 572 can be implemented as a mechanical valve: in the first operating configuration, the valve can be closed, inhibiting flow of both particles 501 and medium 532; while in the second operating configuration, the mechanical valve can be open, allowing flow of both particles 501 and medium 532 to pass freely to outlet 594. In examples, valves can be pneumatically activated. In some examples, block 240 of FIG. 2 or block 340 of FIG. 3 can be performed using an apparatus similar to 500, with pathway 552 for FP2 or FW2. In further examples, a washed stream incoming at port 513 can be paused while trapped particles are discharged at port 594.

### Simplified Principle of Operation

An example of differential action of potential barrier 571 can be explained based on fluid kinetic energy and acoustic energy. As described in context of apparatus 400, acoustic energy gradients can preferentially drive heavier or denser particles towards low energy (node) regions, while lighter or less dense particles are displaced towards higher energy regions. In apparatus 500, the kinetic energy of the incoming fluid (particles 501 and medium 532) can selectively drive a lighter fluid across potential barrier 571, leaving behind particles 501 trapped at trapping site 570.

In the first configuration, maintaining barrier 572 higher than barrier 571 can cause medium 532 to follow a path of lower resistance toward outlet 593. In the second configuration, barrier 572 can be shut off (i.e. set to zero), while barrier 571 is maintained. Accordingly, medium 532 and trapped particles 501 can both be directed to outlet 594 which, in the second configuration, is the energetically preferred path for medium 532.

### Example Trapping Operation

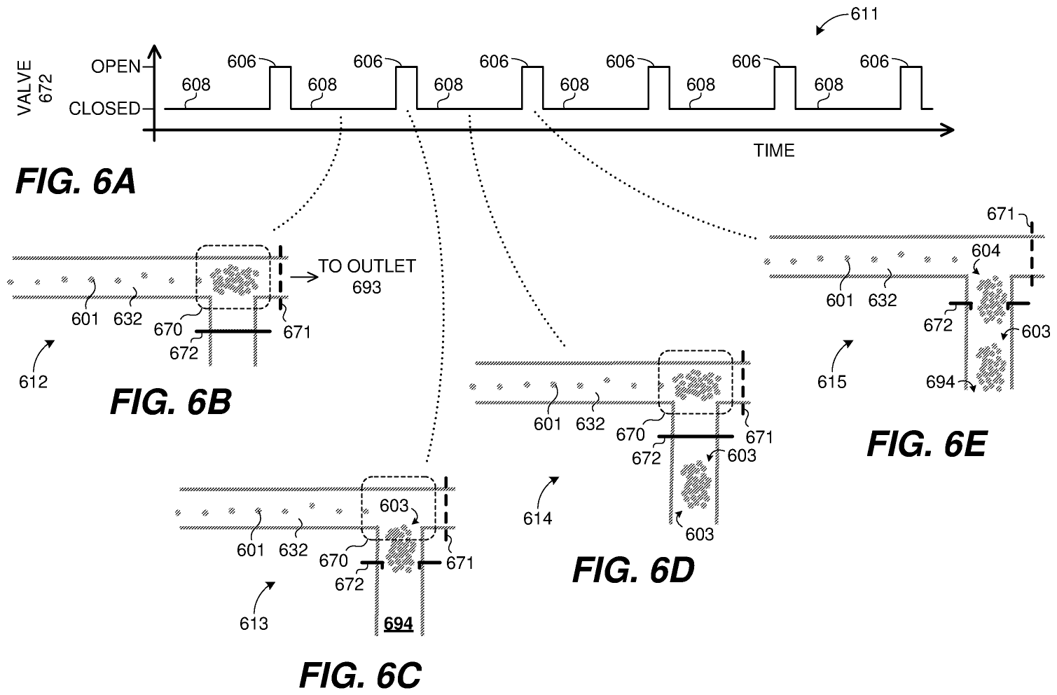

FIG. 6A-6E are diagrams 611-615 illustrating an example trapping operation according to the disclosed technologies. The illustrated trapping operation cycles between an accumulation state and a discharge state.

FIG. 6A is a timing diagram 611 showing accumulation periods 608 interleaved with discharge periods 606 as a control valve 672 is opened or closed. In some examples, valve 672 can be implemented using an acoustophoretic barrier. FIGS. 6B-6E show an exemplary trapping apparatus at four successive phases of operation, as indicated by dotted lines.

FIG. 6B shows a stream of particles 601 in a medium 632 approaching a trapping site 670 during an accumulation period 608, with valve 672 closed. A barrier 671 is configured to provide selective transmission of medium 632, while blocking particles 601 from continuing past barrier 672. FIG. 6C shows a following discharge period 606, with valve 672 open. In this configuration, collected particles 603 are free to exit to outlet 694 due to momentum or pressure of incoming fluid. Barrier 671 is shown unchanged from FIG. 6B to FIG. 6C. In some examples, the outgoing fluid can follow a lower resistance path to outlet 694 even with barrier 671 unchanged, as described herein. In other examples, barrier 671 can be heightened or increased during a discharge period 606. For example, the gradient or amplitude of an acoustic energy barrier 671 can be increased from an accumulation period 608 to a discharge period 606. In further examples, a mechanical valve can be closed in the path from trapping site 670 to outlet 693. Different configurations of mechanical valves and acoustic barriers can be used in accumulation and discharge periods 608, 606 in the channels from trapping site 670 to outlets 693, 694. That is, all or part of valves 671 or 672 can be implemented using acoustic fields generated by one or more transducers similar to 562.

FIG. 6D shows a next accumulation period 608, with valve 672 restored to the configuration of FIG. 6B and a next batch of bioparticles 601 collecting in trap 670, while first batch 603 of bioparticles is on its way downstream. Subsequently, at the next discharge period 606, FIG. 6E shows valve 672 open, similar to FIG. 6B, and a second group of collected particles 604 following group 603 out via outlet port 694. The concentration of particles 603, 604 can be seen to be higher than the concentration of incoming particles 601 in medium 632.

### Example Incubation Apparatus

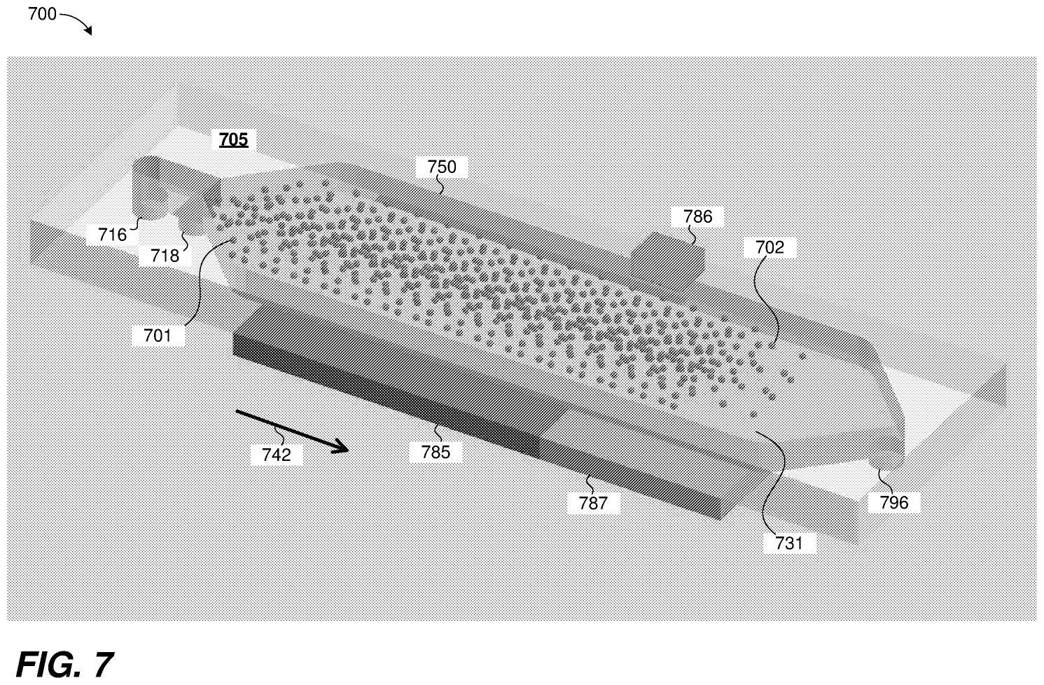

FIG. 7 is a diagram of an example incubation apparatus 700 suitable for the disclosed technologies. Fluid chamber 750 can be formed in a microfluidic substrate 705, and can couple inlet ports 716 and 718 to outlet port 796. Fluid chamber 750 has greater width and greater cross sectional area compared to other fluid pathways of devices 400, 500, or some other microfluidic channels described herein. Consequently, based on conservation of mass flow for series connected microfluidic apparatuses, the longitudinal speed of fluid flow between ports 716, 796 can be greatly reduced compared to the flow speeds in other series connected devices, in inverse proportion to the cross-sectional areas. Thus, dwell time can be greatly increased compared to a narrow channel of comparable length.

A large dwell time can be desirable for incubation, as binding of a label to a bioparticle can require multiple operations, or a binding operation can take tens of minutes. To illustrate, binding of a Zr-89 oxine labeling particle to a cell protein can include a first operation in which the labeling particle can penetrate the cell membrane, and a second operation in which the Zr-89 binds to a target attachment site. The first operation can be fast (under a minute), but can also be easily reversible. Thus, if incubation is terminated with most of the labeling particles inside bioparticle cells, but not bound to target proteins, then, during subsequent washing, many labeling particles can escape through the cell boundary, significantly decreasing the yield of labeled bioparticles. In varying examples, the second operation can occur quickly between 1 second and 1 minute, can take 1-10 minutes, or can take considerably longer from 10 minutes to 2 hours, as labeling particles find their way to attachment sites within cells. In further examples, the time for the second operation can be dependent on the target protein.

In operation, label particles (not shown) in a medium 731 can enter chamber 750 from inlet port 716, while bioparticles 701 can enter chamber 750 from inlet port 718. The mixture can flow slowly in the direction shown by arrow 742. As the fluid progresses toward outlet port 796, bioparticles 701 can gradually be converted to labeled bioparticles 702. The fluid exiting chamber 750 at port 794 can include labeled bioparticles 702 and unbound label particles (not shown). In variations, bioparticles 701 and labeling particles can be mixed upstream of chamber 750 in a mixing apparatus, and the resulting mixture can enter chamber 750 through a single inlet port 716.

A heating and/or cooling assembly 785 can be provided in thermal contact with chamber 750, to provide temperature control. In some examples, temperature can be maintained at a constant, uniform temperature such as 0° C., 4° C., 20° C., or 37° C. Temperature control can be within a predetermined tolerance in a range ±0.01-±2° C., or commonly about ±0.1° C., ±0.3° C., or ±1.0° C. In other examples, controlled temperature variations can be supported, such as a short duration heat flash or a quick freeze. In further examples, controlled spatial variations can be supported, such as a warm incubation in the central portions of fluid chamber 750 and a cool exit temperature proximate to outlet 796. To support temperature or spatial temperature variation, heating and/or cooling assembly 785 can be segmented, or can employ a mix of heating or cooling technologies, such as resistive heaters, lamps, microwave sources, or Peltier coolers.

Fluid chamber 750 can also be equipped with diagnostics. For example, an optical instrument can include a laser 786 arranged to shine light through chamber 750 to a photodetector 787, which can be a single element detector or a pixel array detector. Scattering of light in chamber 750 can be measured and used to estimate density of bioparticles or labeling particles within chamber 750. In varying examples: a single optical instrument can probe density of a single species (e.g. bioparticle, labeling particle); a single optical instrument (e.g. a multi-wavelength source) can probe density of two or more species; or multiple optical instruments can probe respective species or locations within fluid chamber 750.

In some examples, block 260 of FIG. 2 or block 360 of FIG. 3 can be performed using an apparatus similar to 700, with chamber 750 for FC1 or FX1.

### Control of Dwell Time Dispersion

Because laminar flow in a fluid channel can exhibit a speed difference between boundary layers near a sidewall (where flow can be slow) and the center of the channel (where flow can be fast), various techniques can be implemented to mitigate dispersion of dwell time between particles in chamber 750. Particularly, mixing can be used to even out the amount of time different particles spend in slower or faster portions of the flow. In some examples, chamber 750 can be constructed as a series of shorter chambers separated by one or more narrow orifices, each orifice causing peripheral and central portions of the fluid flow to mix. In other examples, topographical features such as diagonal ridges can be used to provide transverse circulation between peripheral and central portions of the flow, even as the particles slowly move longitudinally toward outlet 796. Constrictions and flow control can be provided with stationary physical features in some examples, or dynamically with movable diaphragms and actuators in other examples.

In further examples, mixing can be developed using acoustophoresis. To illustrate, an acoustic transducer can develop a field pattern with a constriction, which can drive particles through the constriction, causing mixing, while a medium can remain generally unaffected by the acoustic field. Acoustic constrictions can be static during an incubation period, can be time varying, or can be spatially varying. A plurality of acoustic transducers can be used. As an illustration, one or more acoustic constrictions can be driven upstream counter to the flow of particles and medium downstream.

In other examples, dispersion can be avoided by operating chamber 750 as a batch mode reactor. As an illustration, chamber 750 can be filled in 10 seconds, maintained in an incubation state for 15 minutes, and evacuated in 10 seconds. Then, the maximum spread in dwell time can be limited to 20 seconds, or ±1.1%.

### Example Labeling Apparatus and System

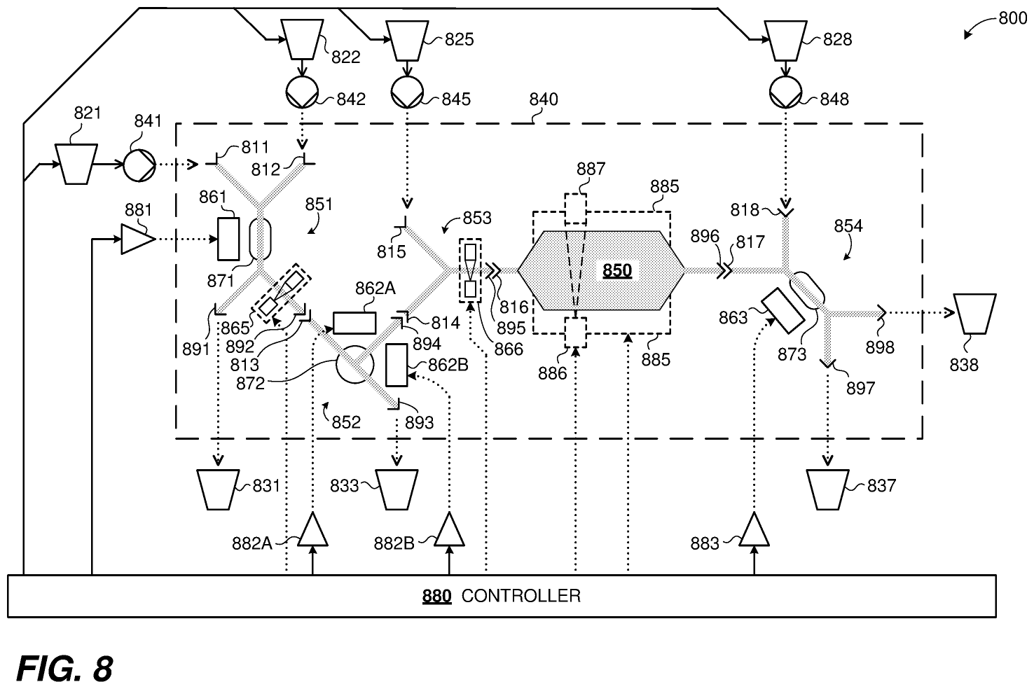

FIG. 8 is a diagram of an example apparatus 840 and system 800 according to the disclosed technologies. Fluid operations described herein can be performed sequentially within labeling apparatus 840. Apparatus can be integrated with one or more fluid reservoirs, fluid chambers, diagnostics, or control equipment to form labeling system 800.

The description begins with apparatus 840, shown in dashed outline. A first section of the apparatus comprises a fluid pathway 851 coupling inlet ports 811, 812 to outlet ports 891, 892. Fluid pathway 851 can be integrated with one or more acoustic transducers 861 creating an acoustic energy pattern with gradients in a region 871. This section of apparatus 840 can operate similarly to washing apparatus 400 described herein. In operation, a bioparticle suspension can be introduced at inlet port 811, and washed bioparticles can be discharged at outlet 892.

A second section of apparatus 840 comprises fluid pathway 852 coupling inlet port 813 to outlet ports 893, 894. One or more acoustic transducers 862A, 862B can create an acoustic energy pattern (at least part of which can be cyclically time varying in some examples) with barriers or gradients in a region 872. This section of apparatus 840 can operate similarly to trapping apparatus 500 described herein. In operation a washed bioparticle suspension introduced from pathway 851 at inlet 813 can be subject to trapping in region 872, where an incoming stream of bioparticles can be accumulated. The accumulated bioparticles can be discharged periodically through outlet 894.

A third section of apparatus 840 comprises fluid pathway 853 coupling inlet ports 814, 815 to outlet port 895. High density bioparticles introduced from pathway 852 at inlet 814 can be combined with labeling particles introduced through inlet 815 to deliver a mixture of bioparticles and labeling particles through outlet 895.

A fourth section of apparatus 840 comprises fluid chamber 850 coupling inlet port 816 to outlet port 896. This section of apparatus 840 can operate similarly to trapping apparatus 700 described herein. A mixture of bioparticles and labeling particles introduced from pathway 853 can be incubated in chamber 850, with labeled bioparticles and excess labeling particles discharged through outlet 896.

A fifth section of apparatus 840 comprises fluid pathway 853 coupling inlet ports 817, 818 to outlet ports 897, 898. Fluid pathway 853 can be integrated with one or more acoustic transducers 863 creating an acoustic energy pattern with gradients in a region 873. This section of apparatus 840 can operate similarly to washing apparatus 400 described herein. In operation, a suspension including labeled bioparticles can be introduced from chamber 850 at inlet port 817, and washed bioparticles can be discharged at outlet 898.

Also shown within the outline of apparatus 840 are some auxiliary devices coupled to chamber 850. One or more heaters and/or coolers 885 can provide temperature control within incubation chamber 850. An optical scattering diagnostic having transmitter 886 and detector 887 can monitor particle within chamber 850. Additional diagnostics 865-866 can be included to monitor particle counts or concentrations at additional locations. In examples, diagnostic 865 can monitor bioparticle counts obtained from washing pathway 851, while diagnostic 866 can monitor counts of bioparticles introduced into chamber 850. Each diagnostic 865, 866 can include a laser light source and photodetector similar to the architecture of optical scattering diagnostic 886-887. Similar diagnostics can be placed near trapping region 872, outlet port 894, or outlet port 896 to respectively monitor bioparticles at the respective locations. Devices 865-866, 885-887 are shown in dashed outline: in varying examples, one or more of devices 885-887 can be included in from apparatus 840; can be provided externally to apparatus 840 as part of system 800; or can be completely omitted. In some examples, chamber 850 can be integrated with one or more other pathways 851-853, 854 e.g. on a microfluidics platform, while in other examples incubation can be performed in a distinct vessel such as a tube or bottle.

The description turns to system 800, which can include apparatus 840 and various other coupled equipment. Bioparticle reservoir 821 can be coupled to inlet port 811, and a collection chamber 838 can be coupled to outlet port 898 to receive labeled bioparticles. Labeling agent reservoir 825 can be coupled to inlet port 815, while wash medium reservoirs 822, 828 can be coupled to inlet ports 812, 818 respectively. Pumps 841, 842, 845, 848 can be provided between reservoirs 821, 822, 825, 828 and their respective inlet ports 811, 812, 815, 818. In varying embodiments, one or more pumps can be integrated with a respective reservoir, can be coupled between the reservoir and the respective inlet port as shown, or can be integrated within apparatus 840. In some examples, a pump (841, 842, 845, or 848) can be pneumatically actuated, e.g. motive force for driving fluids through apparatus 840 can be provided by gas pressure above a reservoir (821, 822, 825, or 828). Waste collection chambers 831, 833, 837 can be coupled to outlet ports 891, 893, 897 respectively as shown.

System 800 can also include drive electronics 881, 882A, 882B, 883 respectively coupled to acoustic transducers 861, 862A, 862B, 863. Each driver 881, 882A, 882B, 883 can include one or more amplifiers and optionally one or more feedback channels for regulation of driven acoustic fields. A controller 880 can provide sequencing and monitoring of various other equipment, such as pumps 841, 842, 845, 848, drive electronics 881, 882A, 882B, 883, or heating, cooling, or monitoring devices 865-866, 885-887.

In some examples, apparatus 840 can be stationary, while in other examples, apparatus can incorporate valves (e.g. membrane valves) and can be quasi-stationary. In further examples, additional moving parts, such as pumps 841, 842, 845, 848 can be integrated into apparatus 840.

Apparatus 840 and system 800 can be configured to support a wide range of applications. In some examples, cells can be labeled with radioligands; magnetic reagents; fluorophores or dyes; proteins; gene components; or gene editing reagents. In additional examples, cells can be labeled with one or more of: drugs, inhibitors, small molecules, cytokines, chemokines, nanoparticles, liposomes, or exosomes. In further examples, cells can be labeled with conjugates of (a) antibodies or antibody fragments and (b) radioisotopes, fluorophores, dyes, drugs, small molecules, nanoparticles, nanotubes or liposomes. In still further examples, cellular proteins can be labeled with adhesion proteins.

### Example Extended Methods

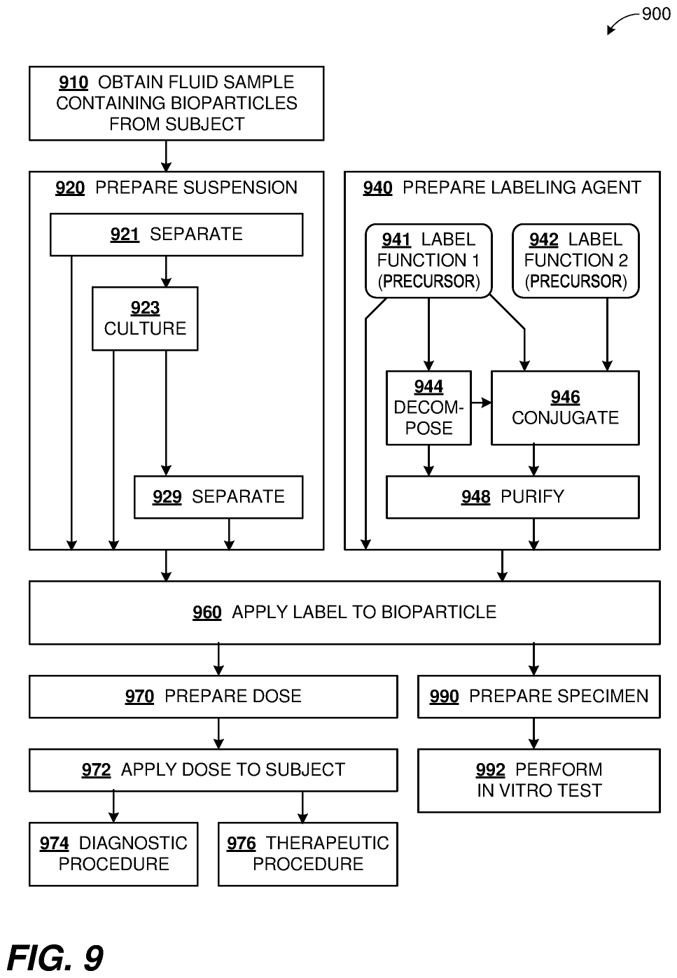

FIG. 9 is a flowchart 900 illustrating examples of extended methods that can be implemented according to the disclosed technologies. These extended methods include derivation or preparation of a sample of target bioparticles to be labeled, derivation or preparation of a sample of labeling particles, a patient procedure using the labeled bioparticles, or an in vitro procedure using the labeled bioparticles.

At the center of FIG. 9, a sample of a label species can be used to tag a sample of a bioparticle species at process block 960. In some examples, process block 960 can incorporate the entire method of FIG. 2, optionally including any variations or extensions described herein. However, in other examples, block 960 can be performed using one or more portions of the method of FIG. 2, or operations described in context of FIG. 4, 5, 6, 7, or 8 herein, singly or in various combinations or subcombinations. In some examples, process block 960 can be performed using the entire apparatus 840 or the entire system 800 described in context of FIG. 8, optionally including any variations or extensions described herein, however this is not a requirement. In other examples, block 960 can be performed using one or more portions of apparatus 840 or system 800, or apparatus described in context of FIG. 2, 4, 5, 6, or 7 herein, singly or in various combinations or subcombinations.

The description of extensions starts with operations associated with preparation of a bioparticle suspension, which can be similar to that provided in reservoir 821. At process block 910, a fluid sample containing bioparticles can be obtained from a subject. To illustrate, the fluid sample can be a blood sample drawn from a human cancer patient. In variations, different types of bodily fluids can be used, or different subjects can be used. Human subjects can be patients, or volunteers in a study or drug trial. Other subjects can include laboratory animals, or other plants or animals. In some examples, samples can be laboratory strains (such as HeLa cells) that can be traced back to a human subject. The sample obtained at block 910 can be used to prepare a bioparticle suspension at process block 920.

In some examples, the fluid sample can contain both desired bioparticles and other constituents (such as, without limitation: antibodies, cell components, other cells, fluids, glucose, hormones, minerals, proteins, or vitamins), and the desired bioparticles can be separated from some or all of the other fluid constituents at process block 921. In some examples, acoustophoretic techniques described herein can be incorporated in block 921. In further examples, centrifugation, aspiration, or other conventional techniques can be used.

In some examples, a product of process block 921 can directly be used as a bioparticle suspension in a labeling procedure. For example, in cases where process block 921 isolates a desired cell, antibody, or fragment, completion of block 921 also completes block 920. In other examples, a separation product can include cells which can be cultured in a growth medium at process block 923. In some examples, block 923 can be performed on a fluidic platform, such as in an apparatus similar to 700 or in a chamber similar to 850. In further examples, a tabletop incubator or another conventional technique can be used. Similar to block 921, in some examples completion of block 923 can also complete block 920, and the cultured bioparticles can be directly used at process block 960. In other examples, cultured bioparticles can be separated from associated byproducts at block 929 to obtain a sample of bioparticles in suspension suitable for labeling. In some examples, a fluidic platform can be used for block 929.

The process pathways shown within block 920 are exemplary. The same or other operations can be combined in various ways according to the needs of a particular application. For example, the blocks can be arranged so that a protein extracted from a cell at block 925 is purified at block 929 and then cleaved at block 927 into a protein fragment; or a molecule can be cleaved multiple times to extract a specific snippet.

The description continues with operations associated with preparation of labeling particles, which can be similar to those provided in reservoir 825. Labeling particles can be prepared at block 940. In some applications, a labeling particle can be produced directly as a label function 941 and used as a labeling particle. An example of such a labeling particle is Zr-89 oxine. In other applications, labeling particles can be obtained from one or more precursors 941 or 942, which can have desired labeling functions. For example, a sample of label precursor 941 can be obtained and decomposed at block 944 to obtain fragments, which can be separated from other byproducts, or purified, at process block 948. Labeling particles such as antibody fragments or adhesion molecule fragments can be obtained in this way.

In further applications, a labeling particle can require synthesis of two or more label functions. Label functions can include attachment functions (e.g. adhesion molecule or fragment; or an antibody or fragment), diagnostic functions (e.g. radioactive, MRI detectable, or fluorescent), or therapeutic functions (e.g. a drug molecule). In such cases, two or more labeling functions 941, 942 can be obtained or synthesized individually, and conjugated at process block 946 to obtain the desired labeling particles. In some examples, conjugation at block 946 can be performed on a product of decomposition block 944. In some examples, it can be desirable to purify the result (e.g. to remove any unreacted excess of functions 942, 944), and purification can be performed at block 948. Label particles such as conjugates of (a) antibody fragments or adhesion molecule fragments with (b) a therapeutic label function or a visualization label function can be obtained in this way.

In some examples, one or more of blocks 944, 946 can be performed on a fluidic platform, such as in an apparatus similar to 700 or in a chamber similar to 850. In further examples, acoustophoretic techniques described herein can be integrated into block 948. The pathway shown in block 940 is exemplary. Conjugation of three or more labeling functions can be performed similarly.

In some examples, preparation of a conjugated labeling particle can itself use an a process similar to that of FIG. 2 or an apparatus or system similar to those of FIG. 8. To illustrate, labeling functions 942, 944 can be provided at reservoirs 821, 825 to an auxiliary copy of apparatus 840, and the conjugated label particles can be obtained at reservoir 898 of that apparatus. In turn, these conjugated labeling particles can be used at process block 960 of the instant method, where they can be provided at reservoir 825 to a main copy of apparatus 840. In additional examples, the two copies of apparatus 840 can be further integrated into a single composite apparatus. In a similar manner, various blocks of block 920 (e.g. blocks 921, 923, 929) can be integrated onto a single platform together with an apparatus similar to 840 used to perform block 960.

The description continues with operations associated with a patient procedure using the labeled bioparticles. At process block 970, a dose can be prepared from the labeled bioparticles. To illustrated, the labeled bioparticles can be transferred to a syringe to prepare an injectable dose. Alternatively, doses suitable for implant, intravenous, oral, or other modes of delivery can be prepared at block 970. Then, at block 972, the dose can be administered to a subject. In varying examples, the subject can be human, another mammal, another animal, or a plant. The subject can be a same subject as the labeled bioparticles were originally obtained from, or a different subject. Dose administration can be by injection, oral, intravenous, implant, or another route. Subsequent to dose administration, a diagnostic procedure can be performed on the subject at block 974, or a therapeutic procedure can be performed at block 976. Diagnostic procedures can include one or more of optical imaging (e.g. for fluorescent labels), MRI, PET, SPECT, fluoroscopy or another diagnostic procedure, and can be performed at one or more times. At block 976, therapy can occur by direct or indirect action of the administered dose in conjunction with living processes or disease processes of the subject. In some examples where a label applied to a bioparticle has a visualization function, therapy 976 can be accompanied by a label-based diagnostic procedure. The labeled bioparticles can be tracked using one or more diagnostic procedures as describe in context of block 974. In other examples, a label can provide a binding or therapeutic function without a visualization function, and therapy 976 can be performed without an accompanying label-based diagnostic.

The description continues with operations associated with an in vitro procedure using the labeled bioparticles. At block 990, a laboratory specimen can be prepared from the labeled bioparticles. To illustrate, the labeled bioparticles can be applied onto an agar culture dish or into a tube containing nutrient broth, either alone or together with another species. In further examples, the specimen can be prepared in an open or closed chamber on a same microfluidic platform or apparatus as a labeling apparatus performing block 960. Then, at block 992, an in vitro test can be performed on the specimen. To illustrate, labeled bioparticles can be tracked over time to determine bioparticle evolution or reactions between the labeled bioparticles and other species in the sample.

The extended methods described herein can be variously combined and are merely illustrative of applications and technology integration possible with the disclosed technologies.

### Example Applications

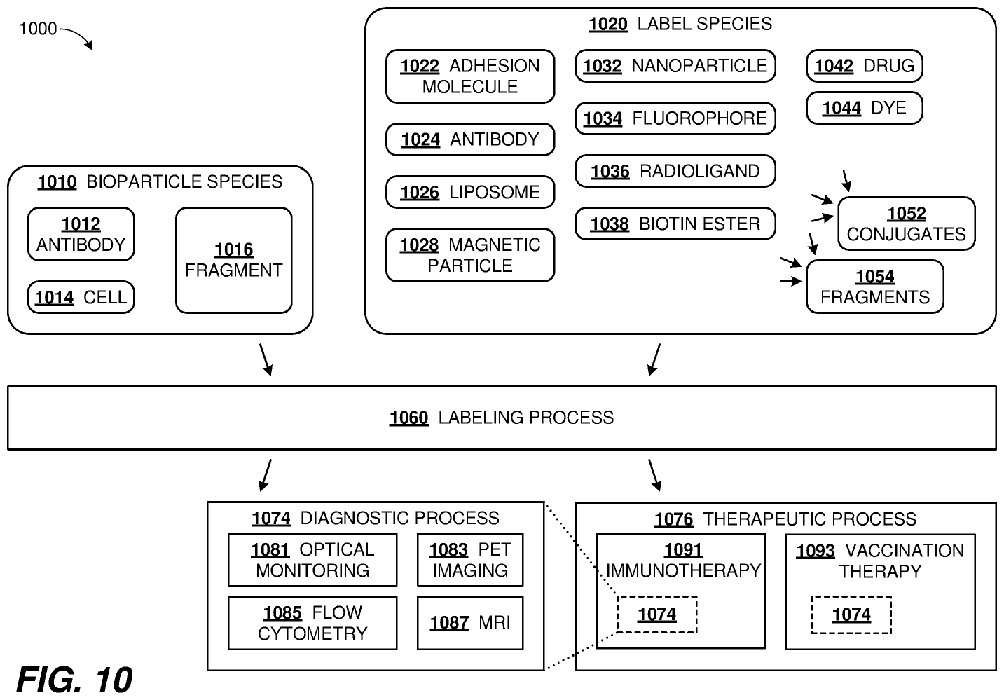

FIG. 10 is a hybrid diagram 1000 illustrating example applications of the disclosed technologies. At the center of FIG. 10, a labeling process 1060 is shown. Process 1060 can be similar to process block 960 and can be performed using the methods or apparatus disclosed herein. In process 1060, a sample of a label species can be used to tag a sample of a bioparticle species, to obtain a sample of labeled bioparticles. Exemplary inputs to labeling process 1060 are shown in the top part of FIG. 10, while exemplary uses for the labeled bioparticles are shown at the bottom of FIG. 10.

Process 1060 can be applied to a wide range of bioparticle species 1010. Some exemplary classes of bioparticles include antibodies 1012 or cells 1014. In further examples, fragmentary bioparticles 1016 can be used, such as antibody fragments or cell components. Various bioparticle species 1010 can be formed using techniques discussed in context of process block 920 herein, or similar techniques.

Similarly, process 1060 can be implemented with a wide range of labeling species 1020. Exemplary classes of labeling species are depicted in FIG. 10, including adhesion molecules 1022, antibodies 1024, liposomes 1026, magnetic particles 1028, nanoparticles 1032, fluorophores 1034, radioligands 1036, biotin esters 1038, drugs 1042, or dyes 1044. Additional classes of labeling particles can be formed by conjugating any of the aforementioned classes with each other (or with another species not represented in FIG. 10). Such conjugated labeling particles are shown generically as conjugates 1052. Further classes of labeling particles can be formed by fragmenting any of the aforementioned classes. Such fragmented labeling particles are shown generically as fragments 1054. In further examples, conjugates 1052 can be formed from fragments 1054, or fragments 1054 can be formed from conjugates 1052. Various labeling species 1020 can be formed using techniques discussed in context of process block 940 herein, or similar techniques.

Turning to uses of labeled bioparticles, a diagnostic process can be performed at process block 1074. Some exemplary diagnostic processes can include optical monitoring 1081, positron emission tomography (PET, or PET imaging) 1083, flow cytometry 1085, or magnetic resonance imaging 1087. In some applications, a given diagnostic process can be performed multiple times, to track evolution of the labeled bioparticle over time, while in other applications two or more different diagnostics can be utilized. Diagnostic process 1074 can be performed in vivo or in vitro.

In further uses, a therapeutic process can be performed at process block 1076. Exemplary classes of therapy include immunotherapy 1091 and vaccination therapy 1093, however other modes of therapy can also be used. Generally, therapy can be accompanied by diagnostic procedures, using information provided by imaging or otherwise detecting the labeled bioparticles to monitor or evaluate therapeutic process 1076. The use of diagnostic procedures in conjunction with therapy is indicated in FIG. 10 by dashed boxes 1074 within therapy processes 1091, 1093.

### Example Performance

Several tests have been performed to verify that the labeling procedures described herein can produce labeled bioparticles that are equivalent to, or can be substituted for, labeled bioparticles produced with conventional techniques.

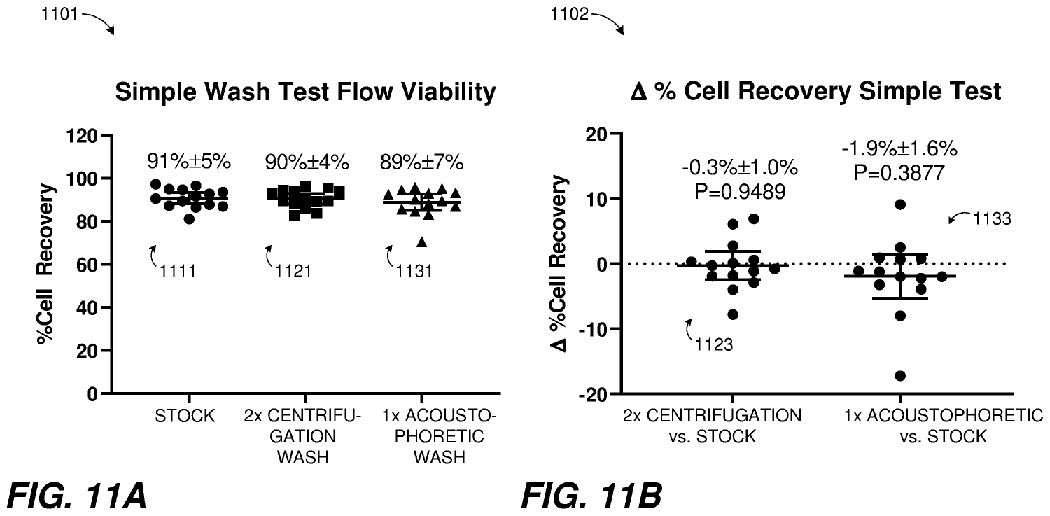

FIG. 11A-11B are charts 1101-1102 comparing performance in a wash test. Stock EL4 cells (murine T cell lymphoma cell line, American Tissue Culture Collection, Manassas VA) were used. In FIG. 11A, viability was measured for 42 cell samples: 14 each for three different conditions. In a left column, circles represent measurements 1111 on cells as obtained from stock, i.e. without any washing. As a baseline, these stock cell samples were measured as having viability of 91%±5%, 91% being the mean of the measurements 1111 and 5% being the standard deviation. The stock cell samples can be similar to stage 111 of FIG. 1, where cells 101 are shown suspended in a culture medium 141.

Another set of 14 measurements was made from the same stock cell supply, after 2 stages of conventional washing with a centrifuge. These measurements 1121 are shown as squares in the middle column of FIG. 11A, and show a viability of 90%±4%. Two conventional washing stages can be similar to the stages of FIG. 1, e.g. initially following stages 111-125, and then repeating stages 117-119 to complete a second washing stage.

Similarly, the right column of FIG. 11A shows measurements 1131 for 14 samples of the same stock cell supply after one stage of an acoustophoretic wash. These measurements 1131 are represented by triangles, and show a viability of 89%±7%. The cell viabilities after either conventional or acoustophoretic washing are comparable to viability in the stock cell supply.

FIG. 11B compares cell recovery in the two wash procedures. Cell losses can occur either through loss of viability or through escape of a cell, e.g. in a waste stream such as from outlet port 491 of FIG. 4. Cell recovery is simply one (or, 100%) minus cell loss. To illustrate, a cell loss of 2% is equivalent to a cell recovery of 98%. Occasional cell recovery values greater than unity can be indicative of measurement errors. Again, for each wash procedure, 14 samples were taken from a stock supply. Viable cell counts were measured before and after washing, and subtracted to obtain cell recovery. In the left column of FIG. 11B, circles denote cell recovery for two stages of a conventional centrifugal wash. These measurements 1123 indicate cell recovery of −0.3%, which is equivalent to cell loss of +0.3%. In the right column of FIG. 11B, circles denote cell recovery for a single stage of an acoustophoretic wash. These measurements 1133 indicate cell recovery of −1.9%, which is equivalent to cell loss of +1.9%. Although greater than the losses of a conventional wash, losses of 2% can be acceptable. The efficiency, speed, or throughput advantages of an acoustophoretic wash process can more than compensate for a small decrease in yield.

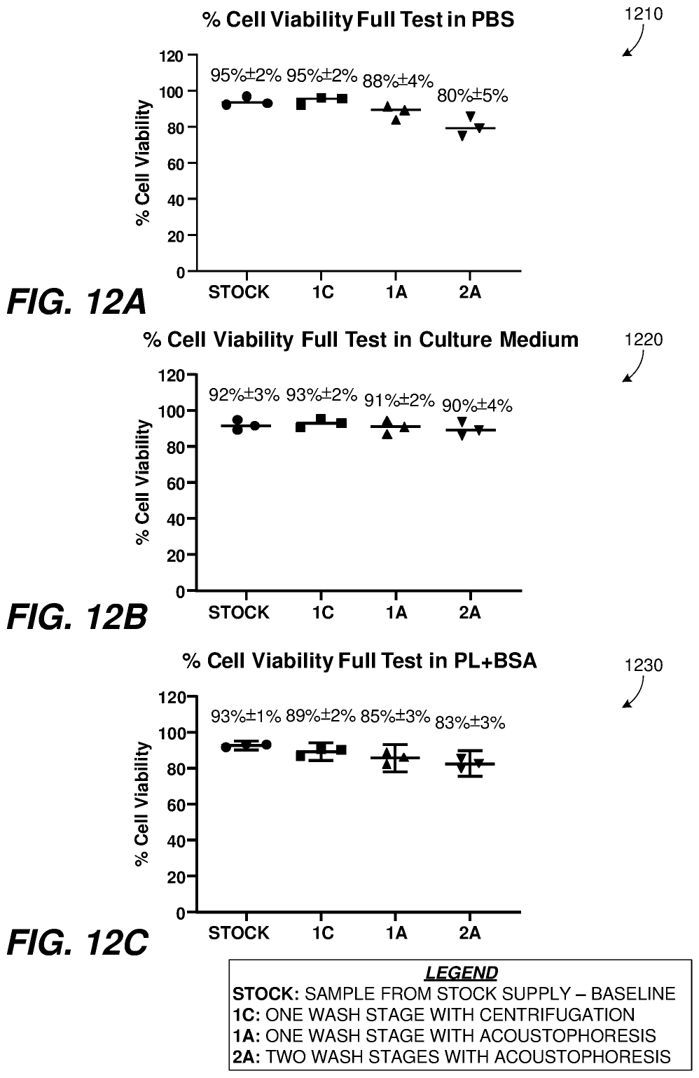

FIGS. 12A-12C are charts 1210, 1220, 1230 comparing performance in labeling tests. Whereas FIGS. 11A-11B show performance for just a wash test, FIGS. 12A-12C show viability results after a complete labeling procedure. Each sample shown started as a stock cell sample, went through a selectable wash procedure, and then incubation with a Zr-89 oxine labeling agent in a conventional table-top incubator. In addition to varying the wash procedure between groups of samples, different wash media were also used. FIG. 12A presents results in a PBS medium, FIG. 12B presents results using a culture medium, and FIG. 12C presents results in a composite medium formed of 96% Plasma-lyte A (Baxter Healthcare Corp., Deerfield IL) and 4% bovine serum albumin (BSA; MilliporeSigma, St. Louis MO). Three experiments were performed for each combination of wash medium and wash procedure shown. In each of FIGS. 12A-12C, four columns of data are presented. A left column shows baseline viability for three stock samples, i.e. without any wash or incubation, while the second column shows viability of incubated and labeled cells using a centrifuge wash procedure similar to that described in context of FIG. 1. The two right-hand columns show results for a single acoustophoresis wash stage (similar to that described in context of FIG. 4) and for two acoustophoresis wash stages, respectively.

Starting with FIG. 12A and PBS medium, viability was found to be 88%±4% and 80%±5% for one and two acoustophoresis wash stages, compared with 95%±2% for each of the stock and centrifugally washed samples. Continuing with FIG. 12B and culture medium, viability was found to be 91%±2% and 90%±4% for one and two acoustophoresis wash stages, compared with 92%±3% for the stock samples and 93%±2% for the centrifugally washed samples. Finally, in FIG. 12C with composite medium, viability was found to be 85%±3% and 83%±3% for one and two acoustophoresis wash stages, compared with 93%±1% for the stock samples and 89%±2% for the centrifugally washed samples. Useable viability yields can be obtained in any of the three media with either one or two stages of acoustophoresis wash.

### Additional Examples

The following are additional examples of the disclosed technologies.

Example 1 is a method, including: (a) washing, using acoustophoresis and from a first medium to a buffer medium distinct from the first medium, bioparticles flowing in a first fluid pathway; (b) trapping the washed bioparticles using acoustophoresis in a second fluid pathway; (c) incubating a mixture of labeling particles and the trapped bioparticles from the trapping in a fluid chamber for a predetermined duration to develop labeled bioparticles; (d) washing the mixture using acoustophoresis in a third fluid pathway; and (e) extracting the labeled bioparticles from the third fluid pathway.

Example 2 includes the subject matter of Example 1, and further specifies that the bioparticles comprise one or more of: antibodies; antibody fragments; cells; cell components; cell membrane components; extracellular vesicles; or proteins.

Example 3 includes the subject matter of Example 2, and further specifies that the bioparticles comprise T cells, regulatory T cells, or dendritic cells.

Example 4 includes the subject matter of Example 2, and further specifies that the bioparticles comprise mammalian cells or human cells.

Example 5 includes the subject matter of Example 2, and further specifies that the bioparticles comprise cellular proteins or cell membrane components.

Example 6 includes the subject matter of Example 2, and further specifies that the bioparticles comprise viable cells or dead cells.

Example 7 includes the subject matter of any of Examples 1-6, and further specifies that the labeling particles comprise one or more of: an adhesion molecule; an adhesion molecule fragment; an antibody; an antibody fragment; a biotin ester; a drug; a dye; a fluorophore; a liposome; an inorganic nanoparticle; a nanotube; a magnetic label; a protein; a radioligand; or a small molecule drug.

Example 8 includes the subject matter of Example 7, and further specifies that the labeling particles comprise a carboxyfluorescein succinimidyl ester (CFSE), a fluorescent dye, a fluorophore conjugated antibody, or a fluorescent expression protein.

Example 9 includes the subject matter of Example 7, and further specifies that the labeling particles comprise an F-19 perfluorocarbon nanoemulsion; or an iron oxide nanoparticle.

Example 10 includes the subject matter of Example 7, and further specifies that the labeling particles comprise a DNA binding dye; a viability dye; or an ion indicator dye.

Example 11 includes the subject matter of Example 7, and further specifies that the labeling particles comprise: Zr-89 oxine, In-111 oxine, or a Zr-89 conjugated antibody.

Example 12 includes the subject matter of any of Examples 1-11, and further specifies: driving the bioparticles suspended in the first medium into the first fluid pathway as a first substream in contact with and in a same direction as a second substream of the buffer medium.

Example 13 includes the subject matter of any of Examples 1-12, and further specifies that the washing the bioparticles comprises applying a first acoustic field gradient to drive the bioparticles from the first medium to the buffer medium.

Example 14 includes the subject matter of any of Examples 1-13, and further specifies: conveying the washed bioparticles from the first fluid pathway into the second fluid pathway while conveying the first medium into a fourth fluid pathway distinct from the second fluid pathway.

Example 15 includes the subject matter of any of Examples 1-14, and further specifies that the trapping includes: applying a second acoustic field to establish one or more potential barriers in the second fluid pathway; and accumulating the bioparticles at the one or more potential barriers while the buffer medium flows past the one or more potential barriers.

Example 16 includes the subject matter of any of Examples 1-15, and further specifies: periodically extracting the trapped bioparticles from the second fluid pathway and conveying the extracted trapped particles to the fluid chamber.

Example 17 includes the subject matter of any of Examples 1-16, and further specifies: combining the bioparticles from the trapping with the labeling particles to form the mixture, wherein the combining is performed in the second fluid pathway, in a seventh fluid pathway en route from the second fluid pathway to the fluid chamber, or in the fluid chamber.

Example 18 includes the subject matter of any of Examples 1-17, and further specifies: during the incubating, controlling a temperature of the mixture to remain within a predetermined range.

Example 19 includes the subject matter of any of Examples 1-18, and further specifies that the incubating includes applying a third acoustic field to enhance mixing between the labeling particles and the bioparticles.

Example 20 includes the subject matter of any of Examples 1-19, and further specifies that the incubating includes applying a third acoustic field to enhance mixing among the bioparticles to reduce dwell time dispersion of the bioparticles in the fluid chamber.

Example 21 includes the subject matter of any of Examples 1-20, and further specifies that the washing the mixture includes: driving a third substream of the mixture in contact with and in a same direction as a fourth substream of a second buffer medium; and applying a fourth acoustic field gradient to the third and fourth substreams to separate unbound labeling particles from the labeled bioparticles.

Example 22 includes the subject matter of any of Examples 1-21, and further specifies that the extracting the labeled bioparticles: conveys the labeled bioparticles into a fifth fluid pathway; and conveys unbound labeling particles into a sixth fluid pathway distinct from the fifth fluid pathway.

Example 23 includes the subject matter of any of Examples 1-22, and further specifies, prior to operation (a): obtaining a fluid sample from a first subject; preparing a suspension of the bioparticles in the first medium from the fluid sample.

Example 24 includes the subject matter of Example 23, and further specifies that the fluid sample comprises first cells and the preparing includes: culturing the first cells in a growth medium to obtain second cells, wherein the bioparticles are the second cells.

Example 25 includes the subject matter of Example 23, and further specifies that the fluid sample comprises antibodies and the preparing includes: separating the antibodies from other components of the fluid sample, wherein the bioparticles are the antibodies.

Example 26 includes the subject matter of Example 23, and further specifies that fluid sample comprises antibodies and the preparing includes: extracting the antibodies from the fluid sample; cleaving the antibodies to obtain antibody fragments, wherein the bioparticles are the antibody fragments.

Example 27 includes the subject matter of Example 23, and further specifies that the first subject is a mammal or a human subject.

Example 28 includes the subject matter of any of Examples 1-27, and further specifies, prior to operation (c): obtaining a sample of a labeling precursor; decomposing the labeling precursor to obtain labeling fragments, wherein the labeling particles are the labeling fragments; and separating the labeling fragments from other products of the decomposing.

Example 29 includes the subject matter of Example 28, and further specifies that the labeling fragments comprise antibody fragments or adhesion molecule fragments.

Example 30 includes the subject matter of any of Examples 1-29, and further specifies, prior to operation (c): obtaining samples of a first labeling precursor and a second labeling precursor; conjugating the first and second labeling precursors to obtain the labeling particles; and purifying the labeling particles.

Example 31 includes the subject matter of Example 30, and further specifies that the labeling particles comprise antibody fragments or adhesion molecule fragments conjugated with a therapeutic label function or a visualization label function.

Example 32 includes the subject matter of any of Examples 1-31, and further specifies, subsequent to operation (e): preparing a dose from the extracted labeled bioparticles; and introducing the dose into a second subject.

Example 33 includes the subject matter of Example 32, and further specifies that the dose is one or more of: a diagnostic or a therapeutic.

Example 34 includes the subject matter of Example 32, and further specifies that the second subject is a mammal or a human subject.

Example 35 includes the subject matter of Example 32, and further specifies that the second subject is the first subject.

Example 36 includes the subject matter of Example 32, and further specifies that the second subject is distinct from the first subject.

Example 37 includes the subject matter of any of Examples 1-36, and further specifies that one or more of operations (a)-(e) are performed within a continuous process.

Example 38 includes the subject matter of any of Examples 1-37, and further specifies that one or more of operations (a)-(e) are performed as a cyclic process.

Example 39 includes the subject matter of any of Examples 1-38, and further specifies that the method is performed as an automated process.

Example 40 includes the subject matter of any of Examples 1-39, and further specifies that the method is performed in a stationary or quasi-stationary apparatus.

Example 41 is an apparatus, including: a first fluid pathway having first and second inlet ports and first and second outlet ports; a first transducer configured to produce a first acoustic field gradient in the first fluid pathway; a second fluid pathway having a third inlet port coupled to the second outlet port, and third and fourth outlet ports; at least one second transducer configured to (a) produce a first acoustic potential barrier in the second fluid pathway and (b) relax the first acoustic potential barrier to a second acoustic field barrier; a third fluid pathway having a fourth inlet port coupled to the fourth outlet port, a fifth inlet port, and a fifth outlet port; a fluid chamber having a sixth inlet port coupled to the fifth outlet port, and a sixth outlet port; a fourth fluid pathway having a seventh inlet port coupled to the sixth outlet port, an eighth inlet port, and seventh and eighth outlet ports; and a third transducer configured to produce a third acoustic field gradient in the fourth fluid pathway.

Example 42 includes the subject matter of any of Examples 41, and further includes: a first substrate upon which two or more of the following are formed: the first fluid pathway; the second fluid pathway; the third fluid pathway; the fluid chamber; or the fourth fluid pathway.

Example 43 includes the subject matter of any of Examples 41-42, and further includes: a second substrate upon which one or more of the following are formed: a first acoustic waveguide coupling the first transducer to the first fluid pathway; a second acoustic waveguide coupling the at least one second transducer to the second fluid pathway; or a third acoustic waveguide coupling the third transducer to the fourth fluid pathway.

Example 44 is a bioparticle labeling system, including: the apparatus according to any one of claims 41-43; a bioparticle reservoir coupled to the first inlet port; a labeling agent reservoir coupled to the fifth inlet port; first and second wash medium reservoirs coupled to the second and eighth inlet ports; a labeled bioparticle collection chamber coupled to the eighth outlet port; waste collection chambers coupled to the first, third, and seventh outlet ports; drivers coupled to the first, second, and third transducers; and a controller coupled to the drivers.

Example 45 includes the subject matter of Example 44, and further specifies that the bioparticle labeling system is configured to label cells with radioligands.

Example 46 includes the subject matter of Example 44, and further specifies that the bioparticle labeling system is configured to label cells with magnetic reagents.

Example 47 includes the subject matter of Example 44, and further specifies that the bioparticle labeling system is configured to label cells with fluorophores, dyes, or proteins.

Example 48 includes the subject matter of Example 44, and further specifies that the bioparticle labeling system is configured to label cells with conjugates of (a) antibodies or antibody fragments and (b) radioisotopes, fluorophores, dyes, drugs, small molecule drugs, nanoparticles, nanotubes or liposomes.

Example 49 includes the subject matter of Example 44, and further specifies that the bioparticle labeling system is configured to label cell membrane components with one or more of: adhesion proteins, receptors, ligands, glycoproteins, or lipids.

Example 50 includes the subject matter of Example 44, and further specifies that the bioparticle labeling system is configured to label cells with one or more of: drugs, inhibitors, small molecule drugs, cytokines, chemokines, nanoparticles, liposomes, biovesicles, or exosomes.

Example 51 includes the subject matter of Example 44, and further specifies that the bioparticle labeling system is configured to label cells with gene components or gene editing reagents.

### A Generalized Computer Environment

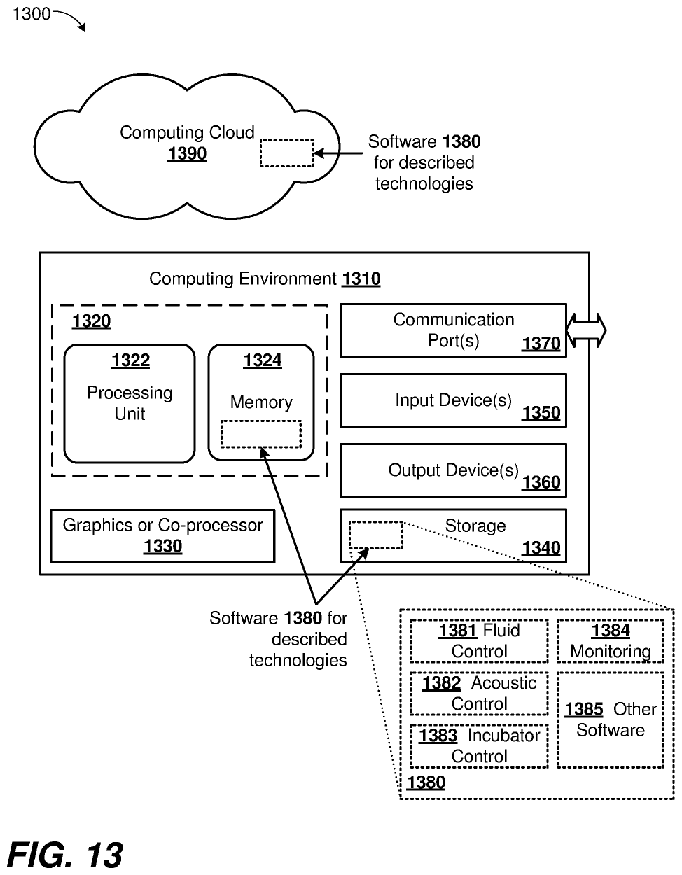

FIG. 13 illustrates a generalized example of a suitable computing system 1300 in which described examples, techniques, and technologies for performing bioparticle labeling, or for operating or configuring an apparatus or system for bioparticle labeling, can be implemented. The computing system 1300 is not intended to suggest any limitation as to scope of use or functionality of the present disclosure, as the innovations can be implemented in diverse general-purpose or special-purpose computing systems. The computing system 1300 can control or monitor a bioparticle labeling process, including washing, trapping, mixing, or incubating stages; or can acquire, process, output, or store operational data.

With reference to FIG. 13, computing environment 1310 includes one or more processing units 1322 and memory 1324. In FIG. 13, this basic configuration 1320 is included within a dashed line. Processing unit 1322 can execute computer-executable instructions, such as for control or data acquisition as described herein. Processing unit 1322 can be a general-purpose central processing unit (CPU), a processor in an application-specific integrated circuit (ASIC), or any other type of processor. In a multi-processing system, multiple processing units execute computer-executable instructions to increase processing power. Computing environment 1310 can also include a graphics processing unit or co-processing unit 1330. Tangible memory 1324 can be volatile memory (e.g., registers, cache, or RAM), non-volatile memory (e.g., ROM, EEPROM, or flash memory), or some combination thereof, accessible by processing units 1322, 1330. The memory 1324 stores software 1380 implementing one or more innovations described herein, in the form of computer-executable instructions suitable for execution by the processing unit(s) 1322, 1330. For example, software 1380 can include software 1381 for controlling fluid handling (e.g. injection, trapping, mixing, or pumps), software 1382 for controlling acoustic devices (e.g. acoustic transducers; acoustic fields, barriers, or gradients; operating sequences for traps), software 1383 for controlling an incubator (e.g. temperature, diagnostics, or batch mode operating sequences), or other software 1384 (including monitoring, pump control, generation of alerts, or a user interface). The inset shown for software 1380 in storage 1340 can be equally applicable to software 1380 elsewhere in FIG. 13. The memory 1324 can also store control parameters, calibration data, measurement data, or database data. The memory 1324 can also store configuration and operational data.

A computing system 1310 can have additional features, such as one or more of storage 1340, input devices 1350, output devices 1360, or communication ports 1370. An interconnection mechanism (not shown) such as a bus, controller, or network interconnects the components of the computing environment 1310. Typically, operating system software (not shown) provides an operating environment for other software executing in the computing environment 1310, and coordinates activities of the components of the computing environment 1310.

The tangible storage 1340 can be removable or non-removable, and can include magnetic disks, magnetic tapes or cassettes, CD-ROMs, DVDs, or any other medium which can be used to store information in a non-transitory way and which can be accessed within the computing environment 1310. The storage 1340 stores instructions of the software 1380 (including instructions and/or data) implementing one or more innovations described herein. Storage 1340 can also store operational sequence data, measurement data, reference data, calibration data, configuration data, sample data, or other databases or data structures described herein.

The input device(s) 1350 can be a mechanical, touch-sensing, or proximity-sensing input device such as a keyboard, mouse, pen, touchscreen, or trackball, a voice input device, a scanning device, or another device that provides input to the computing environment 1310. The output device(s) 1360 can be a display, printer, speaker, optical disk writer, or another device that provides output from the computing environment 1310. Input or output can also be communicated to/from a remote device over a network connection, via communication port(s) 1370.

The communication port(s) 1370 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, acoustic, or other carrier.

A data acquisition system can be integrated into computing environment 1310, either as an input device 1350 or coupled to a communication port 1370, and can include analog-to-digital converters or connections to an instrumentation bus. An instrumentation control system can be integrated into computing environment 1310, either as an output device 1360 or coupled to a communication port 1370, and can include digital-to-analog converters, switches, or connections to an instrumentation bus.

In some examples, computer system 1300 can also include a computing cloud 1390 in which instructions implementing all or a portion of the disclosed technology are executed. Any combination of memory 1324, storage 1340, and computing cloud 1390 can be used to store software instructions and data of the disclosed technologies.

The present innovations can be described in the general context of computer-executable instructions, such as those included in program modules, being executed in a computing system on a target real or virtual processor. Generally, program modules or components include routines, programs, libraries, objects, classes, components, data structures, etc. that perform particular tasks or implement particular data types. The functionality of the program modules can be combined or split between program modules as desired in various embodiments. Computer-executable instructions for program modules can be executed within a local or distributed computing system.

The terms “computing system,” “computing environment,” and “computing device” are used interchangeably herein. Unless the context clearly indicates otherwise, neither term implies any limitation on a type of computing system, computing environment, or computing device. In general, a computing system, computing environment, or computing device can be local or distributed, and can include any combination of special-purpose hardware and/or general-purpose hardware and/or virtualized hardware, together with software implementing the functionality described herein.

### General Considerations

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items. Furthermore, as used herein, the terms “or” and “and/or” mean any one item or combination of items in the phrase.

The systems, methods, and apparatus described herein should not be construed as limiting in any way. Instead, the present disclosure is directed toward all novel and non-obvious features and aspects of the various disclosed embodiments, alone and in various combinations and subcombinations with one another. The disclosed systems, methods, and apparatus are not limited to any specific aspect or feature or combinations thereof, nor do the disclosed systems, methods, and apparatus require that any one or more specific advantages be present or problems be solved. The technologies from any example can be combined with the technologies described in any one or more of the other examples. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus are referred to as “lowest”, “best”, “maximum,” “optimum,” “extremum,” or the like. It will be appreciated that such descriptions are intended to indicate that a selection among a few or among many alternatives can be made, and such selections need not be lower, better, less, or otherwise preferable to other alternatives not considered.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Various of the disclosed methods or operations can be controlled by, or implemented as, computer-executable instructions or a computer program product stored on one or more computer-readable storage media, such as tangible, non-transitory computer-readable storage media, and executed on a computing device (e.g., any available computing device, including tablets, smart phones, or other mobile devices that include computing hardware). Tangible computer-readable storage media are any available tangible media that can be accessed within a computing environment (e.g., one or more optical media discs such as DVD or CD, volatile memory components (such as DRAM or SRAM), or nonvolatile memory components (such as flash memory or hard drives)). By way of example, and with reference to FIG. 13, computer-readable storage media include memory 1324, and storage 1340. The terms computer-readable media or computer-readable storage media do not include signals and carrier waves. In addition, the terms computer-readable media or computer-readable storage media do not include communication ports (e.g., 1370).

Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network, a cloud computing network, or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in Adobe Flash, C, C++, C#, Curl, Dart, Fortran, Java, JavaScript, Julia, Lisp, Matlab, Octave, Perl, Python, Qt, R, Ruby, SAS, SPSS, SQL, WebAssembly, any derivatives thereof, or any other suitable programming language, or, in some examples, markup languages such as HTML or XML, or with any combination of suitable languages, libraries, and packages. Likewise, the disclosed technology is not limited to any particular computer or type of hardware. Certain details of suitable computers and hardware are well known and need not be set forth in detail in this disclosure.

Furthermore, any of the software-based embodiments (comprising, for example, computer-executable instructions for causing a computer to perform any of the disclosed methods) can be uploaded, downloaded, side-loaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, infrared, and optical communications), electronic communications, or other such communication means.

In view of the many possible embodiments to which the principles of the disclosed subject matter may be applied, it should be recognized that the illustrated embodiments are only preferred examples of the disclosed subject matter and should not be taken as limiting the scope of the claims. Rather, the scope of the claimed subject matter is defined by the following claims. We therefore claim all that comes within the scope and spirit of these claims.

1. A method comprising:
(a) washing, using acoustophoresis and from a first medium to a buffer medium distinct from the first medium, bioparticles flowing in a first fluid pathway;
(b) trapping the washed bioparticles using acoustophoresis in a second fluid pathway;
(c) incubating a mixture of labeling particles and the trapped bioparticles from the trapping in a fluid chamber for a predetermined duration to develop labeled bioparticles;
(d) washing the mixture using acoustophoresis in a third fluid pathway; and
(e) extracting the labeled bioparticles from the third fluid pathway.

2. The method according to claim 1, wherein the bioparticles comprise one or more of:
antibodies; antibody fragments; cells; cell components; cell membrane components; extracellular vesicles; or proteins; and
the labeling particles comprise one or more of:
an adhesion molecule; an adhesion molecule fragment; an antibody; an antibody fragment; a biotin ester; a drug; a dye; a fluorophore; a liposome; an inorganic nanoparticle; a nanotube; a magnetic label; a protein; a radioligand; or a small molecule drug.

3-11. (canceled)

12. The method according to claim 1, further comprising:
driving the bioparticles suspended in the first medium into the first fluid pathway as a first substream in contact with and in a same direction as a second substream of the buffer medium;
wherein the washing the bioparticles comprises applying a first acoustic field gradient to drive the bioparticles from the first medium to the buffer medium; and
conveying the washed bioparticles from the first fluid pathway into the second fluid pathway while conveying the first medium into a fourth fluid pathway distinct from the second fluid pathway.

13-14. (canceled)

15. The method according to claim 1, wherein the trapping comprises:
applying a second acoustic field to establish one or more potential barriers in the second fluid pathway; and
accumulating the bioparticles at the one or more potential barriers while the buffer medium flows past the one or more potential barriers; and
the method further comprises:
periodically extracting the trapped bioparticles from the second fluid pathway and conveying the extracted trapped particles to the fluid chamber.

16. (canceled)

17. The method according to claim 1, further comprising:
combining the bioparticles from the trapping with the labeling particles to form the mixture, wherein the combining is performed in the second fluid pathway, in a seventh fluid pathway en route from the second fluid pathway to the fluid chamber, or in the fluid chamber.

18. (canceled)

19. The method according to claim 1, wherein the incubating comprises:
enhancing mixing between the labeling particles and the bioparticles by applying a third acoustic field; or
reducing dwell time dispersion of the bioparticles in the fluid chamber by applying the third acoustic field to enhance mixing among the bioparticles.

20. (canceled)

21. The method according to claim 1, wherein the washing the mixture comprises:
driving a third substream of the mixture in contact with and in a same direction as a fourth substream of a second buffer medium; and
applying a fourth acoustic field gradient to the third and fourth substreams to separate unbound labeling particles from the labeled bioparticles.

22-24. (canceled)

25. The method according to claim 1, further comprising, prior to operation (a):
obtaining a fluid sample comprising antibodies from a subject;
preparing a suspension of the bioparticles in the first medium, the preparing comprising:
(i) separating the antibodies from other components of the fluid sample, wherein the bioparticles are the antibodies; or
(ii) extracting the antibodies from the fluid sample, and cleaving the antibodies to obtain antibody fragments, wherein the bioparticles are the antibody fragments.

26-29. (canceled)

30. The method according to claim 1, further comprising, prior to operation (c):
obtaining samples of a first labeling precursor and a second labeling precursor;
conjugating the first and second labeling precursors to obtain the labeling particles; and
purifying the labeling particles;
wherein the labeling particles comprise (i) antibody fragments or adhesion molecule fragments conjugated with (ii) a therapeutic label function or a visualization label function.

31. (canceled)

32. The method of according to claim 1, further comprising, subsequent to operation (e):
preparing a dose from the extracted labeled bioparticles; and
introducing the dose into a second subject.

33. The method of claim 32, wherein the dose is a therapeutic.

34-37. (canceled)

38. The method according to claim 1, wherein the method is performed as an automated process, or wherein one or more of operations (a)-(e) are performed as a cyclic process.

39. (canceled)

40. The method according to claim 1, wherein the method is performed in a stationary or quasi-stationary apparatus.

41. An apparatus comprising:
a first fluid pathway having first and second inlet ports and first and second outlet ports;
a first transducer configured to produce a first acoustic field gradient in the first fluid pathway;
a second fluid pathway having a third inlet port coupled to the second outlet port, and third and fourth outlet ports;
at least one second transducer configured to (a) produce a first acoustic potential barrier in the second fluid pathway and (b) relax the first acoustic potential barrier to a second acoustic field barrier;
a third fluid pathway having a fourth inlet port coupled to the fourth outlet port, a fifth inlet port, and a fifth outlet port;
a fluid chamber having a sixth inlet port coupled to the fifth outlet port, and a sixth outlet port;
a fourth fluid pathway having a seventh inlet port coupled to the sixth outlet port, an eighth inlet port, and seventh and eighth outlet ports; and
a third transducer configured to produce a third acoustic field gradient in the fourth fluid pathway.

42. The apparatus of claim 41, further comprising a first substrate upon which two or more of the following are formed:
the first fluid pathway; the second fluid pathway; the third fluid pathway; the fluid chamber; or the fourth fluid pathway.

43. The apparatus of claim 41, further comprising a second substrate upon which one or more of the following are formed:
a first acoustic waveguide coupling the first transducer to the first fluid pathway;
a second acoustic waveguide coupling the at least one second transducer to the second fluid pathway; or
a third acoustic waveguide coupling the third transducer to the fourth fluid pathway.

44. A bioparticle labeling system comprising:
the apparatus according to claim 41;
a bioparticle reservoir coupled to the first inlet port;
a labeling agent reservoir coupled to the fifth inlet port;
first and second wash medium reservoirs coupled to the second and eighth inlet ports;
a labeled bioparticle collection chamber coupled to the eighth outlet port;
waste collection chambers coupled to the first, third, and seventh outlet ports;
drivers coupled to the first, second, and third transducers; and
a controller coupled to the drivers.

45. The bioparticle labeling system of claim 44, wherein the bioparticle labeling system is configured to label cells with radioligands.

46. The bioparticle labeling system of claim 44, wherein the bioparticle labeling system is configured to label cells with magnetic reagents.

47. The bioparticle labeling system of claim 44, wherein the bioparticle labeling system is configured to label cells with fluorophores, dyes, or proteins.

48. The bioparticle labeling system of claim 44, wherein the bioparticle labeling system is configured to label cells with conjugates of (a) antibodies or antibody fragments and (b) radioisotopes, fluorophores, dyes, drugs, small molecule drugs, nanoparticles, nanotubes, or liposomes.

49. The bioparticle labeling system of claim 44, wherein the bioparticle labeling system is configured to label cell membrane components with one or more of: adhesion proteins, receptors, ligands, glycoproteins, or lipids.

50. The bioparticle labeling system of claim 44, wherein the bioparticle labeling system is configured to label cells with one or more of: a drug, an inhibitor, a small molecule drug, a cytokine, a chemokine, a nanoparticle, a liposome, a biovesicle, or an exosome.

51. The bioparticle labeling system of claim 44, wherein the bioparticle labeling system is configured to label cells with gene components or gene editing reagents.

52. The apparatus of claim 42, wherein the fluid chamber is formed on the first substrate, and further comprising a thermal regulator, wherein the apparatus is configured to regulate a temperature within the fluid chamber to remain within a predetermined range during an incubation operation.

53. The method according to claim 1, wherein the bioparticles comprise cells and the labeling particles comprise radioligands or iron nanoparticles, or comprise an F-19 reagent.

54. The method according to claim 1, wherein the bioparticles comprise cells and the labeling particles comprise: a DNA binding dye, a viability dye, an ion indicator dye, or a fluorescent expression protein.

55. The method according to claim 1, wherein the bioparticles are antibodies or antibody fragments and the labeling particles comprise: a radioisotope, a fluorochrome, a dye, a drug, a small molecule drug, nanoparticles, nanotubes, or liposomes.

56. The method according to claim 1, wherein the bioparticles are cellular proteins and the labeling particles comprise: adhesion proteins, receptors, or ligands.

57. The method according to claim 1, wherein the bioparticles comprise cells and the labeling particles comprise: a drug, an inhibitor, a small molecule drug, a cytokine, a chemokine, a nanoparticle, a liposome, a biovesicle, or an exosome.

58. The method according to claim 1, wherein the bioparticles comprise cells and the labeling particles comprise: gene components or a gene editing reagent.
